(19) United States
(12) Patent Application Publication
(10) Pub. No.: US 2017/0319540 A1
(43) Pub. Date: Nov. 9, 2017

(54) Methods and Compositions for the Treatment of Non-Alcoholic Steatohepatitis

(71) Applicant: Vivus, Inc., Campbell, CA (US)

(72) Inventor: Craig Peterson, Mountain View, CA (US)

(21) Appl. No.: 15/588,077

(22) Filed: May 5, 2017

Related U.S. Application Data
(60) Provisional application No. 62/332,610, filed on May 6, 2016.

(51) Int. Cl. A61K 31/357; A61K 31/137; A61K 9/00

(52) U.S. Cl. CPC A61K 31/357; A61K 9/0053; A61K 31/137

(57) ABSTRACT

The present disclosure relates to methods of preventing, treating, delaying the onset of, and delaying the progression of liver disease by administering topiramate in combination with phentermine to a patient in need thereof.

### RELATED APPLICATIONS

This application claims priority to U.S. Patent Application No. 62/332,610, filed May 6, 2016, the contents of which are herein incorporated by reference in their entirety.

### INCORPORATION OF SEQUENCE LISTING

The contents of the text file named “VIVU-092-001US_SeqList.txt”, which was created on May 5, 2017 and is 7 KB in size, are hereby incorporated by reference in their entirety.

### FIELD OF INVENTION

The disclosure relates to, among other things, methods for preventing, delaying the onset of, slowing the progression of and/or treating liver disease comprising administering topiramate in combination with phentermine, to a patient in need thereof. Treatment with a combination of phentermine and topiramate results in improvements in multiple factors associated with liver disease, including non-alcoholic steatohepatitis and non-alcoholic fatty liver disease.

### BACKGROUND OF THE INVENTION

Liver disease is generally classified as acute or chronic based upon the duration of the disease. Liver disease may be caused by infection, injury, cancer, exposure to drugs or toxic compounds, alcohol, impurities in foods, and the abnormal build-up of normal substances in the blood, an autoimmune process, a genetic defect (such as haemochromatosis), or unknown cause(s). Common liver diseases include cirrhosis, liver fibrosis, non-alcoholic fatty liver disease (NAFLD), non-alcoholic steatohepatitis (NASH), hepatic ischemia reperfusion injury, primary biliary cirrhosis (PBC), and hepatitis.

Liver disease is a leading cause of death worldwide. The American Liver Foundation estimates that more than 20 percent of the population has NAFLD. When left untreated, NAFLD can progress to NASH, causing serious adverse effects. NASH resembles alcoholic liver disease, but occurs in people who drink little or no alcohol. The major feature in NASH is fat in the liver, along with inflammation and damage. Patients with NASH often feel well and are not aware that they have a liver problem, but NASH can lead to cirrhosis, and permanent liver damage and scarring resulting in impaired liver function.

NASH affects 2 to 5 percent of Americans. An additional 10 to 20 percent of Americans have fat in their liver, but no inflammation or liver damage, a condition called “fatty liver.” Although having fat in the liver is not normal, by itself it may not result in permanent damage. If fat is found in the liver a liver biopsy may be performed to determine if the individual has fatty liver without inflammation and damage (NAFLD) or if the individual has NASH. Individuals with NAFLD are at risk to progress to NASH. NASH can progress to cirrhosis, liver failure and hepatic carcinoma.

Other compounds and methods have been disclosed for treating liver disorders. See for example, U.S. Pat. No. 8962687 and US Patent Pub. No. 20150342943.

The frequency of both NASH and NAFLD is increasing both in the United States and globally, possibly because of high rates of obesity as well as unhealthy diets and sedentary lifestyles. Obesity also contributes to diabetes and high blood cholesterol, which can further complicate the health of someone with NASH. The occurrence of diabetes and high blood cholesterol is also increasing.

NASH is usually first suspected in a person who is found to have elevations in liver tests that are included in routine blood test panels, such as alanine aminotransferase (ALT) or aspartate aminotransferase (AST). When further evaluation shows no apparent reason for liver disease (such as medications, viral hepatitis, or excessive use of alcohol) and when x-rays or imaging studies of the liver show fat, NASH is suspected. A liver biopsy is typically used to make a differential diagnosis between NASH and simple fatty liver, although less invasive methods that utilize Ultrasound Elastography, Magnetic Resonance Elastography, or Liver Scintigraphy are gaining wider acceptance. NASH is diagnosed when examination of the tissue biopsy shows fat along with inflammation and damage to liver cells. If the tissue shows fat without inflammation and damage, NAFLD is diagnosed.

NASH frequently has few or no symptoms. Patients generally feel well in the early stages and only begin to have symptoms—such as fatigue, weight loss, and weakness—once the disease is more advanced or cirrhosis develops. The progression of NASH can take years, even decades. The process can stop and, in some cases, reverse on its own without specific therapy or it can slowly worsen, causing scarring or “fibrosis” to appear and accumulate in the liver. As fibrosis worsens, cirrhosis develops; the liver becomes scarred, hardened, and unable to function normally. Not every person with NASH develops cirrhosis, but once serious scarring or cirrhosis is present, few treatments can halt the progression. About 3.5% of patients with NASH each year will progress to liver cirrhosis (Toshikuni et al. World J Gastroenterol 20(26):8393-8406 (2014)). A person with cirrhosis experiences fluid retention, muscle wasting, bleeding from the intestines, and liver failure. Liver transplantation is the only treatment for advanced cirrhosis with liver failure, and transplantation is increasingly performed in people with NASH. NASH ranks as one of the major causes of cirrhosis in America, behind hepatitis C and alcoholic liver disease.

The underlying cause of NASH is still not clear. It most often occurs in persons who are middle-aged and overweight or obese. Many patients with NASH have elevated blood lipids, such as cholesterol and triglycerides, and many have diabetes or prediabetes, but not every obese person or every patient with diabetes has NASH. Furthermore, some patients with NASH are not obese, do not have diabetes, and have normal blood cholesterol and lipids. NASH can occur without any apparent risk factor and can even occur in children. Thus, NASH is not simply obesity that affects the liver. Factors that are possible candidates for underlying causes of NASH include: insulin resistance, release of toxic inflammatory proteins by fat cells (cytokines), and oxidative stress (deterioration of cells) inside liver cells.

Currently, no specific therapies for NASH exist. Persons with this disease are advised to reduce their weight (if obese or overweight), follow a balanced and healthy diet, increase physical activity, and avoid alcohol and unnecessary medications. People with NASH often have other medical conditions, such as diabetes, high blood pressure, or elevated cholesterol. Experimental approaches under evaluation in patients with NASH include antioxidants, such as vitamin E, selenium, and betaine. These medications act by reducing the oxidative stress that appears to increase inside the liver in patients with NASH. Another experimental approach to treating NASH is the use of newer antidiabetic medications—even in persons without diabetes. Most patients with NASH have insulin resistance, meaning that the insulin normally present in the bloodstream is less effective for them in controlling blood glucose and fatty acids in the blood than it is for people who do not have NASH. Currently available antidiabetic medications make the body more sensitive to insulin and may help reduce liver injury in patients with NASH. Studies of these medications, including rosiglitazone, and pioglitazone, are ongoing.

Topiramate, a sulfamate-substituted monosaccharide with the chemical name 2,3,4,5-bis-O-(1methyletylidene)-β-D-fructopyranose sulfamate, has been reported for use in treating obesity and promoting weight loss, and is also marketed for treating migraine headaches and seizure related disorders. A variety of dosages of topiramate can be used for these purposes, depending on the weight, age, gender, and other characteristics of the subject. Topiramate, 2,3:4,5-bis-O-(1-methylethylidene)-beta-D-fructopyranose sulfamate, was originally described in U.S. Pat. No. 4,513,006, along with its use in treating epilepsy and glaucoma. Topiramate, as Topamax® (Johnson & Johnson Corp.), has been approved by the US FDA as a migraine medication as well as a treatment for epilepsy and glaucoma. Topiramate has also been proposed for use in treating other conditions, such as bipolar disorder, neuropathic pain, impulse control disorders, psoriasis, and amyotrophic lateral sclerosis. See U.S. Pat. Nos. 6,699,840 to Almarsson et al.; 6,323,236 to McElroy et al.; 5,760,006 to Shank et al.; and 5,753,694 to Shank et al.

A formulation of topiramate, in combination with a second agent, phentermine, has been developed and is now commercially available as a medication for the treatment of obesity and potentially related conditions such as type 2 diabetes (QSYMIA®, available from Vivus, Inc., Mountain View, Calif.). Qsymia is currently available as a capsule containing controlled release topiramate beads and phentermine in immediate release form. See U.S. Pat. Nos. 7,056,890, 7,553,818, 7,659,256, and 7,674,776 to Najarian and 8,580,298, 8,580,299, 8,895,057, 8,895,058, 9,011,905 and 9,011,905 to Narjarian et al.

### SUMMARY OF THE INVENTION

Methods are disclosed for treating, slowing progression of and delaying or preventing onset of liver disease. The methods comprise administering to a patient that has been identified as being at risk for liver disease or having been diagnosed with liver disease, a therapeutically effective amount of topiramate in combination with a therapeutically effective amount of phentermine.

In some embodiments of the methods of the disclosure, the patient has a body mass index of at least 27 kg/m². In some embodiments, the patient has a body mass index of at least 30 kg/m².

The liver disease can be any liver disease, including, but not limited to, chronic and/or metabolic liver diseases. In some embodiments, the liver disease is selected from the group consisting of chronic liver disease, metabolic liver disease, steatosis, liver fibrosis, primary sclerosing cholangitis (PSC), cirrhosis, liver fibrosis, non-alcoholic fatty liver disease (NAFLD), non-alcoholic steatohepatitis (NASH), hepatic ischemia reperfusion injury, primary biliary cirrhosis (PBC), and hepatitis.

In some embodiments, the liver disease is steatosis. In additional embodiments, the liver disease is liver fibrosis, or primary sclerosing cholangitis (PSC).

In a preferred aspect the patient has been diagnosed with nonalcoholic fatty liver disease (NAFLD) and topiramate and phentermine are administered to prevent progression to nonalcoholic steatohepatitis (NASH).

In some embodiments of the methods of the disclosure, the topiramate is a compound of formula (I): or a pharmaceutically acceptable salt, isomer, stereoisomer, or tautomer thereof and the phentermine is a compound of formula (II): or a pharmaceutically acceptable salt, isomer, stereoisomer, or tautomer thereof. In some embodiments, the topiramate and the phentermine are administered orally in a single dosage form that is administered once daily. In some embodiments, the topiramate is administered at a daily dose of 23, 46, 69, or 92 mg and the phentermine is administered at a daily dose of 3.75, 7.5 11.25 or 15 mg. In some embodiments, the 3.75, 7.5, 11.25 and 15 mg of phentermine is provided as 4.67, 9.33, 14 and 18.67 mg phentermine hydrochloride. In some embodiments, the patient is administered a first daily dose of 23 mg topiramate and 3.75 mg phentermine for 1 to 3 weeks followed by a second daily dose of 46 mg topiramate and 7.5 mg phentermine for at least 10 weeks. In some embodiments, the second daily dose is administered for at least 56 weeks.

Preferred daily doses include 3.75 mg immediate release phentermine in combination with 23 mg controlled release topiramate (3.75/23), 7.5 mg immediate release phentermine in combination with 46 mg controlled release topiramate (7.5/46), 11.25 mg immediate release phentermine in combination with 69 mg controlled release topiramate (11.25/69), and 15 mg immediate release phentermine in combination with 92 mg controlled release topiramate (15/92).

The combination of phentermine and topiramate may be administered to the patient for a period of weeks, months or years and patients may increase doses over time. In one aspect the patient starts with a daily dose of 3.75/23 for 1 to 2 weeks then increases the dose to 7.5/46. The patient may maintain that dose for 1 to 3 months and then either stay on the 7.5/46 dose for up to 2 years or longer or increase to the 15/92 dose. Some patients, rather than increasing directly to the 15/92 dose from the 7.5/46 dose will take the 11.25/69 dose for a period of 1 to 2 weeks before moving to the 15/92 dose.

In the methods provided herein, the topiramate and phentermine can be co-administered. In such embodiments, the topiramate and phentermine can be administered together as a single pharmaceutical composition, or separately in more than one pharmaceutical composition. Accordingly, also provided herein is a pharmaceutical composition comprising a therapeutically effective amount of topiramate and a therapeutically effective amount of phentermine.

Moreover, the application provides uses of the compounds in the manufacture of a medicament for the treatment of a liver disease. Also provided is a kit that includes topiramate and optionally phentermine. The kit may further comprise a label and/or instructions for use of the topiramate and optionally phentermine, in treating a liver disease in a human in need thereof. Further provided are articles of manufacture that include topiramate and optionally phentermine, and a container. In one embodiment, the container may be a vial, jar, ampoule, preloaded syringe, or an intravenous bag.

The disclosure provides a method of treating or slowing the progression of liver disease in a patient in need thereof, comprising administering to the patient a therapeutically effective amount of topiramate in combination with a therapeutically effective amount of phentermine, wherein the liver disease is non-alcoholic steatohepatitis (NASH) and wherein the therapeutically effective amount of topiramate is selected from 46 mg per day and 92 mg per day and the therapeutically effective amount of phentermine is selected from 7.5 mg per day and 15 mg per day wherein the 7.5 mg phentermine is provided as 9.33 mg phentermine hydrochloride and the 15 mg phentermine is provided as 18.66 mg phentermine hydrochloride.

The disclosure provides a method of delaying the progression from NAFLD to NASH in a patient, comprising: identifying a patient having NAFLD and administering to the patient an oral dosage form comprising immediate release phentermine and controlled release topiramate. In some embodiments, the oral dosage form comprises: a) 3.75 mg immediate release phentermine in combination with 23 mg controlled release topiramate, orb) 7.5 mg immediate release phentermine in combination with 46 mg controlled release topiramate, or c) 11.25 mg immediate release phentermine in combination with 69 mg controlled release topiramate, or d) 15 mg immediate release phentermine in combination with 92 mg controlled release topiramate. In some embodiments, the oral dosage form is administered to the patient for at least 3 months. In some embodiments, a first oral dosage form is administered to the patient for 1 to 2 weeks and a second oral dosage form is administered to the patient for 3 months and wherein the first oral dosage form comprises 3.75 mg immediate release phentermine in combination with 23 mg controlled release topiramate and the second oral dosage form comprises 7.5 mg immediate release phentermine in combination with 46 mg controlled release topiramate. In some embodiments, the step of identifying a patient having NAFLD comprises determining that the patient has at least one of the following: (i) hepatic accumulation of triglycerides in the hepatocytes in the absence of clinically significant alcohol intake; (ii) simple hepatic steatosis; (iii) fat making up more than 10% of the weight of the liver, and (iii) fibrosis. In some embodiments, the method further comprises continuing to administer the oral dosage form to the patient following the onset of NASH and thereby reducing the severity of NASH symptoms in the patient. In some embodiments, including those wherein the method further comprises continuing to administer the oral dosage form to the patient following the onset of NASH and thereby reducing the severity of NASH symptoms in the patient, administering prevents the onset of NASH in the patient for the at least 3 months. In some embodiments, the progression of NAFLD to NASH is prevented if the patient has at least one of the following after the at least 3 months: (i) percentage of liver weight that is contributed by fat has not increased since starting the administration; (ii) the fibrotic area of the liver has not increased since starting the administration (iii) the inflammation area of the liver has not increased since starting administration and (iv) NAFLD activity score is less than 5. In some embodiments, the method further comprises achieving in the patient a reduction in the patient's NAFLD activity score to less than 5. In some embodiments, the method further comprises achieving in the patient an improvement in at least one of the following: a decreased NAFLD activity score, decreased plasmas AST, decreased liver tryiglyceride levels, decreased plasma ALT, and decreased whole blood glucose. In some embodiments, the method further comprises achieving in the patient an improvement in at least one of the following over the measurements for the patient prior to administering the oral dosage form to the patient: a decreased NAFLD activity score, decreased plasmas AST, decreased liver tryiglyceride levels, decreased plasma ALT, and decreased whole blood glucose. In some embodiments, the patient is identified as potentially having NAFLD and being at risk to progress to NASH if the patient is obese, has type 2 diabetes or insulin resistance.

The disclosure provides a method of delaying the progression from NASH to liver cirrhosis in a patient, comprising: identifying a patient having NASH and administering to the patient an oral dosage form comprising: a) 3.75 mg immediate release phentermine in combination with 23 mg controlled release topiramate, orb) 7.5 mg immediate release phentermine in combination with 46 mg controlled release topiramate, or c) 11.25 mg immediate release phentermine in combination with 69 mg controlled release topiramate, or d) 15 mg immediate release phentermine in combination with 92 mg controlled release topiramate.

### BRIEF DESCRIPTION OF THE DRAWINGS

FIG. 1 shows body weight after the start of treatment with vehicle, PTHN/TPM or telmisartan for the STAM model mice.

FIG. 2 shows food consumption after the start of treatment with vehicle, PTHN/TPM or telmisartan for the STAM model mice.

FIG. 3A shows body weight after the start of treatment with vehicle, PTHN/TPM or telmisartan for the STAM model mice.

FIG. 3B shows liver weight after the start of treatment with vehicle, PTHN/TPM or telmisartan for the STAM model mice.

FIG. 3C shows liver to body weight ratios after the start of treatment with vehicle, PTHN/TPM or telmisartan for the STAM model mice.

FIG. 4A shows whole blood glucose levels after the start of treatment with vehicle, PTHN/TPM or telmisartan for the STAM model mice.

FIG. 4B shows plasma ALT levels after the start of treatment with vehicle, PTHN/TPM or telmisartan for the STAM model mice.

FIG. 4C shows plasma AST levels after the start of treatment with vehicle, PTHN/TPM or telmisartan for the STAM model mice.

FIG. 4D shows liver triglyceride levels after the start of treatment with vehicle, PTHN/TPM or telmisartan for the STAM model mice.

FIG. 5A shows NAFLD activity scores after the start of treatment with vehicle, PTHN/TPM or telmisartan for the STAM model mice.

FIG. 5B shows fibrosis area after the start of treatment with vehicle, PTHN/TPM or telmisartan for the STAM model mice.

FIG. 5C shows fat deposition area after the start of treatment with vehicle, PTHN/TPM or telmisartan for the STAM model mice.

FIG. 5D shows inflammation area after the start of treatment with vehicle, PTHN/TPM or telmisartan for the STAM model mice.

FIG. 6 shows gene expression analysis for TNF-α, TIMP-1, Alpha-SMA and MMP-9 after the start of treatment with vehicle, PTHN/TPM or telmisartan for the STAM model mice.

FIG. 7 shows gene expression analysis for collagen type 1 and MCP-1 after the start of treatment with vehicle, PTHN/TPM or telmisartan for the STAM model mice.

### DETAILED DESCRIPTION

In this application, including the appended claims, the singular forms “a,” “an,” and “the” are often used for convenience. However, it should be understood that these singular forms include the plural unless otherwise specified. It should also be understood that all patents, publications, journal articles, technical documents, and the like, referred to in this application, are hereby incorporated by reference in their entirety and for all purposes.

Unless otherwise defined, all terms used in this application should be given their standard and typical meanings in the art, and are used as those terms would be used by a person of ordinary skill in the art at the time of the invention.

“Active agent” as used herein encompass not only the specified molecular entity but also its pharmaceutically acceptable, pharmacologically active analogs, including, but not limited to, salts, esters, amides, prodrugs, conjugates, active metabolites, and other such derivatives, analogs, and related compounds as will be discussed infra. Therefore, reference to “phentermine,” for example, encompasses not only phentermine per se but also salts and other derivatives of phentermine, e.g., phentermine hydrochloride. It is to be understood that when amounts or doses are specified, that those amounts or doses refer to the amount or dose of active agent per se and not to a salt or the like. For example, when it is indicated that a dose or amount of phentermine is 7.5 mg, which would correspond to 9.33 mg phentermine hydrochloride and not 7.5 phentermine hydrochloride. The molecular weight of phentermine is 149.23 g/mol and the molecular weight of phentermine hydrochloride is 185.69 g/mol. So, for example, 3.75 mg phentermine is provided by 4.67 mg phentermine hydrochloride.

“Administering” as used herein includes to any route of administration, for example, oral, parenteral, intramuscular, transdermal, intravenous, inter-arterial, nasal, vaginal, sublingual, subungal, etc. Administering can also include prescribing a drug to be delivered to a subject, for example, according to a particular dosing regimen, or filling a prescription for a drug that was prescribed to be delivered to a subject, for example, according to a particular dosing regimen.

“Body Mass Index” or “BMI” as used herein is an index of weight-for-height that is commonly used to classify overweight and obesity in adults. BMI may be calculated by multiplying an individual's weight, in kilograms, by height, in meters. Currently the CDC and WHO define obesity as having a BMI of 30 or higher. A BMI between 25 and 29.9 is considered overweight. A BMI over 40 is sometimes characterized as morbidly obese. Individuals having a BMI between 30 and 35 may also be referred to as moderately obese, from 35 to 40 severely obese and over 40 very severely obese.

A “daily dose” of a particular material refers the amount of the material administered in a day. A daily dose can be administered as a single dose or as multiple doses. When a daily dose is administered as multiple doses, the daily dose is the sum of the amount of material administered in all of the multiple doses that are administered over the course of one day. For example, a daily dose of 12 mg can be administered in a single 12 mg dose once per day, in 6 mg doses administered twice per day, in 4 mg doses administered three times per day, in 2 mg doses administered six times per day, etc. The multiple doses can be the same or different doses of the material, unless otherwise specified. When a daily dose is administered as multiple doses, the multiple doses can be administered by the same or different route of administration, unless otherwise specified. Thus, a daily dose of 12 mg can include, for example, a 10 mg intramuscular dose and a 2 mg oral dose administered over the course of one day.

The term “dosage form” denotes any form of a pharmaceutical composition that contains an amount of active agent sufficient to achieve a therapeutic effect with a single administration. When the formulation is a tablet or capsule, the dosage form is usually one such tablet or capsule, although this is not required unless otherwise specified. The frequency of administration that will provide the most effective results in an efficient manner without overdosing will vary with the characteristics of the particular active agent, including both its pharmacological characteristics and its physical characteristics, such as hydrophilicity.

The term “controlled release” refers to a drug-containing formulation or fraction thereof in which release of the drug is not immediate, i.e., with a “controlled release” formulation, administration does not result in immediate release of the drug into an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained release, modified release and delayed release formulations. That is, “controlled release” includes “sustained release” (synonymous with “extended release”), referring to a formulation that provides for gradual release of an active agent over an extended period of time, as well as “delayed release,” indicating a formulation that, following administration to a patient, provides for a measurable time delay before the active agent is released from the formulation into the body of the patient.

Administration of one compound “with” a second compound, as used herein, includes but is not limited to cases where the two compounds are administered simultaneously or substantially simultaneously. For example, administration of a first compound with a second compound can include administering the first compound in the morning and administering the second compound in the evening, as well as administering the first and second compounds in the same dosage form or in two different dosage forms that at the same or nearly the same time.

“Topiramate” as used herein includes not only the chemical compound 2,3,4,5-bis-O-(1methyletylidene)-β-D-fructopyranose sulfamate, but also all stereoisomers, such as enantiomers and diasteriomers, thereof, as well as salts, mixed salts, polymorphs, solvates, including mixed hydrates and mixed solvates, of one or more stereoisomers or mixtures of stereoisomers. The molecular formula is C₁₂H₂₁NO₈S and the molecular weight is 339.4 g/mol.

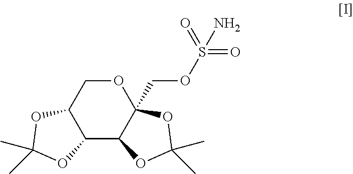 [SMILES: CC1(C)O[C@@H]2[C@@H](CO[C@@]3(COS(N)(=O)=O)OC(C)(C)O[C@@H]23)O1]

In preferred aspects of the present invention topiramate may be administered to a patient in a single daily dosage of 15 mg/day to 30 mg/day, e.g., 23 mg/day, of topiramate for an initial period, for example 2 to 4 weeks. Next, the dose may be increases to a dosage of 35 mg/day to 55 mg/day, e.g., 46 mg/day, of topiramate. The patient may remain on that second dose for months, for example 2 to 4 months or longer. Optionally the dose may be increased again after 2 to 4 months to a dosage of 60 mg/day to 80 mg/day, e.g., 69 mg/day, of topiramate for another shorter period, for example 2-4 weeks and then increased to a dosage of 85 mg to 125 mg/day, e.g., 92 mg/day of topiramate for the next 2-4 months or longer depending on the duration of treatment needed.

“Phentermine” as used herein includes not only the chemical compound 2-methyl-1-phenylpropan-2-amine, but also all stereoisomers, such as enantiomers and diasteriomers, thereof, as well as salts, mixed salts, polymorphs, solvates, including mixed hydrates and mixed solvates, of one or more stereoisomers or mixtures of stereoisomers. The molecular formula is C₁₀H₁₅N HCl and its molecular weight is 185.7 (hydrochloride salt) or 149.2 (free base).

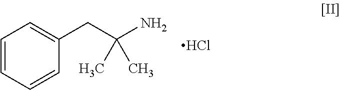

The term “sustained release” (synonymous with “extended release”) is used in its conventional sense to refer to a drug formulation that provides for gradual release of a drug over an extended period of time, and that preferably, although not necessarily, results in substantially constant blood levels of a drug over an extended time period. The term “delayed release” is also used in its conventional sense, to refer to a drug formulation which, following administration to a patient provides a measurable time delay before drug is released from the formulation into the patient's body.

The terms “treating” and “treatment” include the following actions: (i) preventing a particular disease or disorder from occurring in a subject who may be predisposed to the disease or disorder but has not yet been diagnosed as having it; (ii) inhibiting the disease, i.e., arresting its development; or (iii) relieving the disease by reducing or eliminating symptoms and/or by causing regression of the disease.

The terms “effective amount” and “therapeutically effective amount” of a pharmacologically active agent refers to an amount that is nontoxic and effective for producing a therapeutic effect upon administration to a subject.

The term “unit dosage forms” as used herein refers to physically discrete units suited as unitary dosages for the individuals to be treated. That is, the compositions are formulated into discrete dosage units each containing a predetermined, “unit dosage” quantity of an active agent calculated to produce the desired therapeutic effect in association with the required pharmaceutical carrier. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent, e.g., two tablets or capsules taken together may provide a therapeutically effective dosage of topiramate, such that the unit dosage in each tablet or capsule is approximately 50% of the therapeutically effective amount.

A suitable daily dose of phentermine is in the range of 3 mg to 30 mg. For example, 3 mg, 5 mg, 8 mg, 10 mg, 12 mg, 15 mg, 20 mg, 25 mg, 30 mg, or the like is administered to a patient as a daily dosage. In another example, 3.75 mg, 7.5 mg, 11.25 mg and 15 mg or the like is administered to a patient as a daily dosage. Each of the aforementioned “daily dosages” is generally although not necessarily administered as a single daily dose.

Daily doses of PHEN/TPM ER that available include 3.75 mg phentermine with 23 mg topiramate extended-release, 7.5 mg phentermine with 46 mg topiramate extended-release, 11.25 mg phentermine with 69 mg topiramate extended-release, and 15 mg phentermine with 92 mg topiramate extended-release.

The patient may receive a specific dosage of PHEN/TPM ER over a period of weeks, months, or years, e.g., 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years and the like. In some embodiments the patient starts at one dose for a period of time and then increases doses after a period.

In one embodiment, a daily dose of phentermine can be administered with one or more of daily dose of about 23 mg topiramate, the daily dose of about 46 mg topiramate, the daily dose of about 69 mg topiramate, and the daily dose of about 92 mg topiramate. In another embodiment, a daily dose of about 3.75 mg of phentermine can be administered with the daily dose of about 23 mg of topiramate. In yet another embodiment, a daily dose of about 7.5 mg of phentermine can be administered with the daily dose of about 46 mg of topiramate. In still another embodiment, a daily dose of about 11.25 mg phentermine can be administered with the daily dose of about 69 mg of topiramate. In a further embodiment, a daily dose of about 15 mg of phentermine can be administered with the daily dose of about 92 mg of topiramate.

In a particular embodiment, phentermine can be administered in an immediate release form. In a specific embodiment, the topiramate can be administered in a controlled release form. In other embodiments, the controlled release form is a polymer coated bead. In an additional embodiment, phentermine can be administered in an immediate release form and the topiramate can be administered in a controlled release form. In some embodiments, the phentermine and the topiramate are administered in a single unit dosage form having a controlled release topiramate portion and an immediate release phentermine portion.

The “NAFLD Activity Score” or “NAS” is a scoring system developed for use in clinical trials. The scoring system includes 14 histological features, 4 of the features are evaluated semi-quantitatively and 9 are evaluated as present or absent. The scoring system is described in Kleiner et al. Hepatology, Vol 41, No. 6, 1313-1321 (2005). Five features were observed to be independently associated with a diagnosis of NASH in adult biopsies: steatosis, hepatocellular ballooning, lobular inflammation, fibrosis and the absence of lipogranulomas. The NAS reported by Kleiner et al. may be used in both adults and children with any degrees of NAFLD and shows reasonable inter-rater reproducibility. A NAS of greater than or equal to 5 correlates with a diagnosis of NASH and biopsies with scores of less than 3 are diagnoses as “not NASH”.

Other scoring systems can also be used to assess the disease level of a patient or to assess improvement in the patient following treatment. These systems include the Brunt score (Brunt et al. Am J Gastroenterol 94:2467-2474 (1999)), the NAFLD fibrosis score and the BARD score (Vuppalanchi and Chalasani, Hepatology:49:306-317(2009)).

The term “unit dosage forms” as used herein refers to physically discrete units suited as unitary dosages for the individuals to be treated. That is, the compositions are formulated into discrete dosage units each containing a predetermined, “unit dosage” quantity of an active agent calculated to produce the desired therapeutic effect in association with the required pharmaceutical carrier. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent, e.g., two tablets or capsules taken together may provide a therapeutically effective dosage of topiramate, such that the unit dosage in each tablet or capsule is approximately 50% of the therapeutically effective amount.

By “pharmaceutically acceptable” is meant a material that is not biologically or otherwise undesirable, i.e., the material may be incorporated into a pharmaceutical formulation administered to a patient without causing any undesirable biological effects or interacting in a deleterious manner with any of the other components of the composition in which it is contained. When the term “pharmaceutically acceptable” is used to refer to a pharmaceutical excipient, it is implied that the carrier or excipient has met the required standards of toxicological and manufacturing testing and/or that it is included on the Inactive Ingredient Guide prepared by the U.S. Food and Drug administration.

“Pharmacologically active” (or simply “active”) as in a “pharmacologically active” analog, refers to a compound having the same type of pharmacological activity as the parent compound and approximately equivalent in degree.

As used herein, the term “patient” or “individual” or “subject” refers to any person or mammalian subject for whom or which therapy is desired, and generally refers to the recipient of the therapy to be practiced according to the invention.

“Steatosis” refers to the buildup of fat in the liver. Excessive amounts of triglycerides and other fats can collect inside liver cells for a variety of reasons. In general if the liver comprises about 5-10% fat by weight the patient may be diagnosed as having NAFLD.

“Steatohepatitis” refers to the combination of a buildup of fat in the liver in combination with inflammation of the liver. When not associated with excessive alcohol intake it is referred to as nonalcoholic steatohepatitis (NASH) which is the progressive form of NAFLD. Both NASH and NAFLD can progress to cirrhosis, characterized by failure of the liver to function properly and often resulting in fluid buildup.

Treatment with PHEN/TPM ER may be used as a method for slowing progression, delaying onset of, or treating a liver disease; preventing, slowing, delaying, or reversing progression from NAFLD to NASH and to cirrhosis; preventing, slowing progression, delaying, or treating complications of liver disease, including fibrosis and cirrhosis; by administering to a patient a combination of phentermine and topiramate.

A series of clinical trials were performed to evaluate the efficacy of the combination of phentermine and topiramate for weight loss. The studies included over 4,500 overweight or obese patients treated with PHEN/TPM ER for up to 2 years. The results demonstrated durable weight loss. In addition, beneficial effects on biomarkers that correlate with NAFLD and NASH were also observed.

In a separate analysis, weight loss as a consequence of PHEN/TPM ER therapy was also observed to be accompanied by a decrease in progression to type 2 diabetes (see US Patent Pub. No. 20150099801). In that analysis the treatment was accompanied by an increase in insulin sensitivity, as manifested by reduced glucose and insulin values, and improvements in cardiometabolic risk factors (blood pressure, waist circumference, triglycerides, and HDL-C). Furthermore, systemic inflammation, as measured by hs-CRP and fibrinogen at week 56, was reduced, and levels of the insulin-sensitizing adipocytokine, adiponectin, at week 56, were increased. Since insulin resistance, dyslipidemia, inflammation, and dysregulated secretion of adipocytokines are all hallmarks of cardiometabolic disease, these findings are indicative of the potential reversal of this pathophysiologic process. The patients in the study were obese and lost weight during the study so it was not possible to determine if the improvements in liver markers were independent of weight loss. However, topiramate alone has been shown to improve insulin sensitivity and decreases insulin resistance independent of weight loss so it is possible that the effect is at least partially weight loss independent.

Exemplary liver diseases include, but are not limited to, cirrhosis, liver fibrosis, non-alcoholic fatty liver disease (NAFLD), non-alcoholic steatohepatitis (NASH), hepatic ischemia reperfusion injury, primary biliary cirrhosis (PBC), and hepatitis, including both viral and alcoholic hepatitis. Primary sclerosing cholangitis (PSC) is another example of liver disease.

NAFLD is the build-up of extra fat in liver cells that is not caused by alcohol or viral infection. It is normal for the liver to contain some fat, but if more than 5-10% of the liver's weight is fat than it is called a fatty liver (steatosis). NAFLD may progress and cause the liver to become inflamed (i.e. steatohepatitis), which in turn may cause scarring (i.e. cirrhosis) over time and may lead to liver cancer or liver failure. NAFLD is characterized by the accumulation of fat in hepatocyes and is often associated with some aspects of metabolic syndrome (e.g. type 2 diabetes mellitus, insulin resistance, hyperlipidemia, hypertension). NAFLD often has no symptoms but may be associated with fatigue, weakness, weight loss, loss of appetite, nausea, abdominal pain, spider-like blood vessels, yellowing of the skin and eyes (jaundice), itching, fluid buildup and swelling of the legs (edema) and abdomen (ascites), and mental confusion (particularly as the disease progresses to more severe indications such as NASH). The frequency of this disease has become increasingly common due to consumption of carbohydrate-rich and high fat diets. A subset (about 20%) of NAFLD patients progresses to the development of nonalcoholic steatohepatitis (NASH). NASH is characterized by macrovesicular steatosis, balloon degeneration of hepatocytes, and/or inflammation ultimately leading to hepatic scarring (i.e. fibrosis). Patients diagnosed with NASH may also progress to advanced stage liver fibrosis and eventually cirrhosis. Once NASH is developed, it can cause the liver to undergo destructive remodeling leading to scarring (i.e. cirrhosis) over time. The current treatment for cirrhotic NASH patients with end-stage disease is liver transplant.

NAFLD is the most common chronic liver disease in Europe and the US with a prevalence of up to 30%. It is associated with obesity and diabetes and is considered by many to be the hepatic manifestation of the metabolic syndrome. NAFLD can be characterized by triglyceride accumulation in hepatocyes, while NASH may be associated with fibrosis and progression to cirrhosis or hepatocellular carcinoma. Liver fibrosis is the excessive accumulation of extracellular matrix proteins, including collagen that occurs in most types of chronic liver diseases. Advanced liver fibrosis results in cirrhosis, liver failure, and portal hypertension and often requires liver transplantation. In some cases, advanced liver fibrosis may result in liver cancer.

Another common liver disease is primary sclerosing cholangitis (PSC). It is a chronic or long-term liver disease that slowly damages the bile ducts inside and outside the liver. In patients with PSC, bile accumulates in the liver due to blocked bile ducts, where it gradually damages liver cells and causes cirrhosis, or scarring of the liver. Currently, there is no effective treatment to cure PSC. Many patients having PSC ultimately need a liver transplant due to liver failure, typically about 10 years after being diagnosed with the disease. PSC may also lead to bile duct cancer.

Liver disease may be diagnosed by a number of methods with liver biopsy being the most frequently used. The minimal diagnostic criteria for liver disease include the presence of >5% macrovesicular steatosis, inflammation and liver cell ballooning. Liver biopsy does have some drawbacks as it is an invasive procedure associated with severe complications in 0.3%-3.0% of cases, and leads to death in 0.01% of cases. Moreover, it evaluates only a small sample of the liver, which may not be representative.

Non-invasive methods such as biomarkers and imaging have also been used to diagnose liver disease. There are a number of biomarkers and diagnostic panels for diagnosing NASH and advanced liver fibrosis. Inflammation, oxidative stress and apoptosis are associated with NASH and markers associated with each have been used. For a review see Filozof et al. Drugs 75:1373-1392 (2015) and in particular page 1377. Non-invasive methods for diagnosis can also be used to diagnose different liver diseases. Imaging studies such as ultrasonography, computed tomography, and magnetic resonance imaging can also be used to detect steatosis, but may not be able to distinguish steatosis from NASH. Techniques including magnetic resonance-based elastigraphy, acoustic radiation force impulse ultrasonography, and liver scintography provide more sensitive methods for differentiating NASH and more advanced fibrosis from simple steatosis.

The presence of active liver disease can be detected by the existence of elevated enzyme levels in the blood. Specifically, blood levels of alanine aminotransferase (ALT) and aspartate aminotransferase (AST), above clinically accepted normal ranges, are known to be indicative of on-going liver damage and are frequently used as surrogate markers for liver injuries. Both are associated with hepatocellular damage even in the absence of histological signs of lobular inflammation. Additionally, blood bilirubin levels or other liver enzymes may be used as detection or diagnostic criteria. Routine monitoring of liver disease patients for blood levels of ALT and AST is used clinically to measure progress of liver disease while on medical treatment. Reduction of elevated ALT and AST to within the accepted normal range is taken as clinical evidence reflecting a reduction in the severity of the patient's on-going liver damage.

Other surrogate markers that may be used in diagnosing liver disease include the ratio of total cholesterol to HDL. High serum triglyceride levels and low serum HDL levels are common in patients with NAFLD. Patients with dyslipidemia that are attending lipid clinics have been estimated to have NAFLD at a rate of 50% (see Assy et al. Dig Dis Sci. 2000:45(10):1929-34. There have been numerous reports indicating that the presence of T2DM is associated with a two to four-fold increase in serious liver disease, cirrhosis and hepatocellular carcinoma (Lomanaco et al. Drugs 2013:73(1):1-14).

Insulin resistance (IR) is thought to play a major role in the pathogenesis of NAFLD and is a key factor in the initiation and perpetuation of NASH (Cusi et al. Gastroenterology 142(4):711-725 e6 (2012). One hypothesis is that triglycerides in the hepatocytes accumulate because of central obesity and IR. The IR leads to enhanced lipolysis which in turn increases circulating free fatty acids and their uptake by the liver. Increased free fatty acids in the liver are combined with impaired hepatic fatty acid metabolism. The accumulation of lipids in the liver exacerbates IR by interfering with the tyrosine phosphorylation and signaling potential of cellular insulin receptor substrates. Progression from steatosis to steatohepatitis and fibrosis may be the result of fat accumulation in the liver resulting in up regulation of hepatocyte apoptosis, mitochondrial dysfunction with increase in reactive oxygen species leading to lipid peroxidation of cell membranes and indication of pro-inflammatory genes such as TNFα and COX-2, which in turn induce additional inflammatory mediators with pro-fibrotic effects. Increased secretion of adipocytokines (leptin, resisten) and pro-inflammatory markers from the adipose tissue in connection with IR and decreased levels of anti-inflammatory cytokines such as adiponectin leads to apoptosis, necroinflammation and fibrosis in the hepatocytes. Immune responses to lipid peroxidation products may also be involved in the disease progression.

The pathogenesis of NAFLD is thought to be related with insulin resistance (IR) syndrome and oxidative stress; the latter resulting from mitochondrial fatty acids (FFAs) oxidation, nuclear factor-kappaB (NFκB)-dependent inflammatory cytokine expression and adipocytokines may promote hepatocellular damage, inflammation, fibrosis and progressive liver disease.

Adipocytokines and other recognized cytokines produced partially by inflammatory cells infiltrating adipose tissue, play an important role in the pathogenesis of IR and NAFLD, through complex and interactive paracrine and endocrine mechanisms. Some adipokines, including adiponectin and leptin decrease IR., while others, including tumor necrosis factor (TNF)-α, interleukin (IL)-6 and resistin enhance IR. The multi-hit hypothesis provides a model that summarizes the complex factors and interactions leading from adipocytokines, FFAs metabolism and IR to NAFLD.

Methods of treating and/or preventing liver disease in a patient in need thereof are disclosed. They include administering to the patient a therapeutically effective amount of an anticonvulsant sulfamate derivative (e.g. topiramate), in combination with a therapeutically effective amount of a sympathomimetic agent (e.g. phentermine).

The results of the present application indicate that anticonvulsant sulfamate derivatives like topiramate in combination with sympathomimetic amines like phentermine may inhibit, prevent, reduce, or reverse liver fibrogenesis. This suggest that a combination of topiramate and phentermine may be used as an anti-fibrotic agent that would have therapeutic or prophylactic effects for treating liver fibrosis such as NASH or PSC. Also, the results described herein suggest that treatment with topiramate and phentermine would lead to improvements in metabolic parameters associated with NASH.

Moreover, the results of the present application indicate that treatment with a combination of topiramate and phentermine, may inhibit, prevent, or reduce the cross-linking of hepatic collagen, liver fibrogenesis, and/or reversal of fibrosis. The present application suggests that, under certain conditions, the combination of an anticonvulsant sulfamate derivative and a sympathomimetic agent would inhibit, reduce, prevent, or reverse biliary fibrosis and portal hypertension. Without being bound to any hypothesis, a combination therapy comprising an anticonvulsant sulfamate derivative and a sympathomimetic agent may impact non-overlapping profibrogenic pathways. Accordingly, the combination therapy comprising an anticonvulsant sulfamate derivative (such as topiramate) and a sympathomimetic agent (such as phentermine) would provide potential therapeutic effects to liver disease.

In certain embodiments, the liver disease is a chronic liver disease. Chronic liver diseases involve the progressive destruction and regeneration of the liver parenchyma, leading to fibrosis and cirrhosis. In general, chronic liver diseases can be caused by viruses (such as hepatitis B, hepatitis C, cytomegalovirus (CMV), or Epstein Barr Virus (EBV)), toxic agents or drugs (such as alcohol, methotrexate, or nitrofurantoin), a metabolic disease (such as non-alcoholic fatty liver disease (NAFLD), non-alcoholic steatohepatitis (NASH), haemochromatosis, or Wilson's Disease), an autoimmune disease (such as Autoimmune Chronic Hepatitis, Primary Biliary Cirrhosis, or Primary Sclerosing Cholangitis), or other causes (such as right heart failure). In one embodiment, the present application provides a method of treating liver fibrosis. In some embodiments, the present application provides a method of treating non-alcoholic steatohepatitis (NASH). In certain embodiment, the present application provides a method of treating primary sclerosing cholangitis (PSC).

In one embodiment, provided herein is a method for reducing the level of cirrhosis. In one embodiment, cirrhosis is characterized pathologically by loss of the normal microscopic lobular architecture, with fibrosis and nodular regeneration. Methods for measuring the extent of cirrhosis are well known in the art. In one embodiment, the level of cirrhosis is reduced by about 5% to about 100%. In one embodiment, the level of cirrhosis is reduced by at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, at least about 95%, or about 100% in the subject.

In certain embodiments, the liver disease is a metabolic liver disease. In one embodiment, the liver disease is non-alcoholic fatty liver disease (NAFLD). NAFLD is associated with insulin resistance and metabolic syndrome (obesity, combined hyperlipidemia, diabetes mellitus (type II) and high blood pressure). NAFLD is considered to cover a spectrum of disease activity, and begins as fatty accumulation in the liver (hepatic steatosis).

It has been shown that both obesity and insulin resistance probably play a strong role in the disease process of NAFLD. In addition to a poor diet, NAFLD has several other known causes. For example, NAFLD can be caused by certain medications, such as amiodarone, antiviral drugs (e.g., nucleoside analogues), aspirin (rarely as part of Reye's syndrome in children), corticosteroids, methotrexate, tamoxifen, or tetracycline. NAFLD has also been linked to the consumption of soft drinks through the presence of high fructose corn syrup which may cause increased deposition of fat in the abdomen, although the consumption of sucrose shows a similar effect (likely due to its breakdown into fructose). Genetics has also been known to play a role, as two genetic mutations for this susceptibility have been identified.

If left untreated, NAFLD can develop into non-alcoholic steatohepatitis (NASH), which is the most extreme form of NAFLD, a state in which steatosis is combined with inflammation and fibrosis. NASH is regarded as a major cause of cirrhosis of the liver of unknown cause. Accordingly, provided herein is a method of treating and/or preventing nonalcoholic steatohepatitis (NASH) in a patient in need thereof, comprising administering to the patient a therapeutically effective amount of topiramate in combination with a therapeutically effective amount of phentermine.

Also provided herein is a method of treating and/or preventing liver fibrosis in a patient in need thereof, comprising administering to the patient a therapeutically effective amount of an Anticonvulsant sulfamate derivative, optionally in combination with a therapeutically effective amount of a Sympathomimetic agent. Liver fibrosis is the excessive accumulation of extracellular matrix proteins including collagen that occurs in most types of chronic liver diseases. In certain embodiments, advanced liver fibrosis results in cirrhosis and liver failure. Methods for measuring liver histologies, such as changes in the extent of fibrosis, lobular hepatitis, and periportal bridging necrosis, are well known in the art.

In one embodiment, the level of liver fibrosis, which is the formation of fibrous tissue, fibroid or fibrous degeneration, is reduced by more that about 90%. In one embodiment, the level of fibrosis, which is the formation of fibrous tissue, fibroid or fibrous degeneration, is reduced by at least about 90%, at least about 80%, at least about 70%, at least about 60%, at least about 50%, at least about 40%, at least about 30%, at least about 20%, at least about 10%, at least about 5% or at least about 2%.

In one embodiment, the compounds provided herein reduce the level of fibrogenesis in the liver. Liver fibrogenesis is the process leading to the deposition of an excess of extracellular matrix components in the liver known as fibrosis. It is observed in a number of conditions such as chronic viral hepatitis B and C, alcoholic liver disease, drug-induced liver disease, hemochromatosis, auto-immune hepatitis, Wilson disease, primary biliary cirrhosis, sclerosing cholangitis, liver schistosomiasis and others. In one embodiment, the level of fibrogenesis is reduced by more that about 90%. In one embodiment, the level of fibrogenesis is reduced by at least about 90%, at least about 80%, at least about 70%, at least about 60%, at least about 50%, at least 40%, at least about 30%, at least about 20%, at least about 10%, at least about 5% or at least 2%.

In still other embodiments, provided herein is a method of treating and/or preventing primary sclerosing cholangitis (PSC) in a patient in need thereof, comprising administering to the patient a therapeutically effective amount of topiramate in combination with a therapeutically effective amount of phentermine.

In some other embodiments, a method is provided for providing a prophalatic treatment of liver disease (including chronic liver disease, a metabolic liver disease, nonalcoholic fatty liver disease), nonalcoholic steatohepatitis (NASH), or liver fibrosis primary sclerosing cholangitis (PSC) in a patient in need thereof, comprising administering to the patient a therapeutically effective amount of topiramate in combination with a therapeutically effective amount of phentermine. In certain other embodiments, a method is provided for providing prophalatic treatment of liver disease (including chronic liver disease, a metabolic liver disease, nonalcoholic fatty liver disease), nonalcoholic steatohepatitis (NASH), or liver fibrosis primary sclerosing cholangitis (PSC) in a patient in need thereof, comprising administering to the patient a therapeutically effective amount of topiramate in combination with a therapeutically effective amount of phentermine. In some embodiment, the prophalatic treatment is provided to the patients having NASH or PSC. In some other embodiment, the effect of prophalatic treatment may be determined by steatosis, fibrosis progression, fasting blood glucose levels, AUC insulin levels, fasting insulin levels, ALT levels, AST levels, cholesterol levels, AUC glucose levels, relative hydroxyproline levels, fibrillar collagen synthesis, and/or body weight.

In certain embodiments, a method is provided for treating pre-existing abnormal levels of steatosis, fibrosis progression, fasting blood glucose, AUC insulin, fasting insulin, ALT, AST, cholesterol, AUC glucose, relative hydroxyproline, percent liver fat by weight and/or fibrillar collagen synthesis in a patient in need thereof, comprising administering to the patient a therapeutically effective amount of topiramate in combination with a therapeutically effective amount of phentermine. The abnormal levels may be determined by the levels that are higher than those detected in healthy individuals. In certain other embodiment, the abnormal levels of steatosis, fibrosis progression, fasting blood glucose, AUC insulin, fasting insulin, ALT, AST, cholesterol, AUC glucose, relative hydroxyproline, fibrillar collagen synthesis, and/or percent liver fat by weight are associated with type 2 diabetes mellitus. Methods for measuring the levels or extent of steatosis, fibrosis progression, fasting blood glucose, AUC insulin, fasting insulin, ALT, AST, cholesterol, AUC glucose, relative hydroxyproline, fibrillar collagen synthesis, and/or percent liver fat by weight are well known in the art. In one embodiment, the level or extent of steatosis, fibrosis progression, fasting blood glucose, AUC insulin, fasting insulin, ALT, AST, cholesterol, AUC glucose, relative hydroxyproline, fibrillar collagen synthesis, and/or percent liver fat by weight would be reduced by about 5% to about 80%. In other embodiment, the level or extent of steatosis or fatty liver would be reduced by about 5% to about 80%.

In a further embodiment, provided herein is a method of preventing progression from NAFLD to NASH or cirrhosis in a patient in need thereof, comprising administering to the patient a therapeutically effective amount of topiramate in combination with a therapeutically effective amount of phentermine.

In some embodiments, provided herein is a method for treating or preventing liver damage or injury in a patient in need thereof, comprising administering to the patient a therapeutically effective amount of topiramate in combination with a therapeutically effective amount of phentermine. In some other embodiments, the liver damage or injury may be acute or chronic. In certain embodiments, the acute liver damage or injury may be caused by alcoholic injury or drug overdosing. In certain other embodiment, the liver damage or injury is acetaminophen (APAP) hepatotocity. In other embodiments, the methods for treating or preventing acute liver damage or injury in a patient in need thereof comprising administering to the patient a therapeutically effective amount of topiramate in combination with a therapeutically effective amount of phentermine. In some embodiment, the methods for treating or preventing acute liver alcoholic injury, drug overdosing, or APAP hepatotoxicity in a patient in need thereof, comprising administering to the patient a therapeutically effective amount of topiramate in combination with a therapeutically effective amount of phentermine.

The daily dose of, phentermine, can be any appropriate daily dose. For example, the daily dose of the sympathomimetic agent, for example, phentermine, can be from about 2 mg to about 1,500 mg, for example, a daily dose of about 2 mg to about 20 mg. The daily dose of the sympathomimetic agent can be increased if and when the daily dose of topiramate is increased, although this is not required unless otherwise specified. The ratio of topiramate to phentermine in the different daily doses may be constant, for example, if the first daily dose is 23 mg of topiramate and 3.75 mg phentermine, for a weight of phentermine to topiramate ratio of about 16% (i.e. the weight of the phentermine is about 16% of the weight of the topiramate), then one or more of the second, third, and fourth daily doses can also have about a 16% weight ratio of phentermine to topiramate. Other ratios may also be used, for example, about 10-20%, about 13-17%. The ratio may be maintained for one or more of the second, third and fourth doses. For example, the second daily dose may be about 7.5 mg phentermine and 46 mg topiramate, the third may be about 11.25 mg phentermine and about 69 mg topiramate and the fourth about 15 mg phentermine and about 92 mg topiramate, each daily dose having a ratio of about 16% (the weight of phentermine being about 16% of the weight of phentermine).

Subjects who are candidates to maintain either the daily dose of 46 mg topiramate with 7.5 mg phentermine or the daily dose of 92 mg topiramate with 15 mg phentermine can maintain that regimen. Subjects who respond to the 7.5 mg phentermine/46 mg topiramate daily dose may continue taking that daily dose after the 3 month period for an additional period of time, for example, for 3, 6, 9, 12, 18, 24, or 36 additional months or more.

The phentermine and topiramate used in the dosing regimens and methods described herein can be administered in any suitable dosage form, depending on the desired route of administration. For example, tablets, capsules, caplets, elixirs, syrups, sachets, granules, powders, pellets, and beads are all suitable for oral administration. Dosage forms for these and other routes and modes of administration are discussed, for example, in Remington: The Science and Practice of Pharmacy, which is hereby incorporated by reference in its entirety.

Topiramate can be present in a controlled release dosage form, such as a sustained release form, a delayed release form, or a dosage form with both delayed and sustained release. Controlled release forms can be any controlled release form, and can be prepared by any preparation method known in the art. Some controlled release forms include topiramate dispersed within a matrix of one or more controlled release polymers, for example, one or more hydrolyzable or degradable polymers, such as one or more hydrophilic polymers. Other controlled release forms include a topiramate containing dosage form coated with one or more controlled release polymers. Exemplary hydrophilic polymers useful for this purpose include cellulose polymers, such as one or more of hydroxypropyl cellulose, hydroxyethyl cellulose, hydroxypropyl methyl cellulose, methyl cellulose (METHOCEL™), ethyl cellulose, cellulose acetate, cellulose acetate phthalate, and sodium carboxymethylcellulose, acrylic polymers and copolymers, such as polymers or copolymers of one or more of (meth)acrylic acid, methyl(meth)acrylate, and ethyl(meth)acrylate, vinyl polymers and copolymers, such polymers with one or more of polyvinyl pyrrolidone (POVIDONE™ and POVIDONE™ K30), polyvinyl acetate, and ethylene vinyl acetate.

Controlled release dosage forms, such as sustained release dosage forms, can also include additional excipients such one or more binders, diluents, bulking agents, glidents, lubricant, taste-modifying agents, flavorings, colorings, and the like. Such agents can be useful in the manufacturing process of the controlled release dosage form, commercially beneficial, for example, to provide a commercially desirable appearance, taste, or both. Many examples of such excipients are known in the art, and are discussed, for example, in Remington: The Science and Practice of Pharmacy, which is hereby incorporated by reference in its entirety.

Specific examples of controlled release dosage forms of topiramate include polymer matrices that contain the topiramate and a controlled release polymer, tablets coated with a controlled release polymer, osmotic tablets, and polymer coated beads. In one aspect controlled release topiramate beads are made using an extrusion spheronization process to produce a matrix core comprised of topiramate, 40.0% w/w; microcrystalline cellulose (AVICEL™ PH102), 56.5% w/w; and METHOCEL™ A15 LV, 3.5% w/w. The topiramate cores are then coated with ethyl cellulose, 5.47% w/w, and Povidone K30, 2.39% w/w. Such dosage forms can be prepared by methods known in the art, for example, methods described in U.S. Pat. Pub. No. 2009/0304785, which is hereby incorporated by reference.

In one example the composition of the topiramate beads may be 36.85% w/w topiramate, 52.05% w/w microcrystalline cellulose, 3.22% w/w methylcellulose, 5.47% w/w ethyl cellulose, and 2.39% w/w polyvinylpyrrolidone (PVP).

The phentermine can be present in the same dosage form as the topiramate or in a different dosage form. When the phentermine is in a different dosage form from the topiramate, the type of dosage form used for the phentermine can be the same or different from the type of dosage form used for the topiramate. For example, topiramate can be present in a capsule and phentermine can be present in a solution. In that example, the topiramate can be administered orally and the phentermine can be administered intra-muscularly. As another example, the topiramate and phentermine can be present in the same dosage form, such as a powder, bead, or granule, or in the same unit dosage form, such as a capsule, or tablet. When present in a tablet form the tablet can be a multilayer table, for example, a bilayer tablet having an immediate release portion containing the phentermine and a sustained release portion containing the topiramate. A tablet-in-tablet formulation can also be used, where the core comprises a therapeutically effective amount of topiramate that is surrounded by a layer comprising a therapeutically effective amount of phentermine. The topiramate and phentermine can be in direct contact or may be separated by a barrier layer. The core can contain both topiramate and one or more pharmaceutically acceptable excipients. The tablet can be coated with a rapidly dissolving coating or film.

The phentermine can be administered in an immediate release form dosage form. An exemplary immediate release form is an inert bead, such as a non-pareil or sugar sphere, coated with the phentermine, to form one or more coated beads. Additional coating agents, such as film-formers, diluents, plasticizers, binders, coating aids, adhesion aids, and the like, can also be present in the coating of the phentermine coated beads. Further, additional coating layers, such as film-coats or topcoats, can be present either on top of the phentermine coating or between the inert bead and the phentermine coating. Phentermine coated beads can be, for example, mixed with one or more tableting excipients, such as one or more binders, lubricants, glidant, etc., and compressed into one or more tablets. Phentermine coated beads can also be prepared as one or more capsules, for example, by filling one or more capsule shells, such as gelatin capsule shells, with the phentermine coated beads. In one aspect, phentermine hydrochloride is coated onto sugar spheres to provide immediate release phentermine beads. These beads are combined with the topiramate beads described above and then encapsulated into each of a plurality of capsules, with each capsule containing 3.75 mg phentermine (as 4.92 mg phentermine HCl) and 23 mg topiramate or 7.5/46, 11.25/69 and 15/92.

When phentermine is in the same dosage form as the topiramate, such as a unit dosage form with topiramate and phentermine, the unit dosage form can contain a controlled release portion of topiramate and an immediate release portion of phentermine. For example, one or more polymer coated beads containing topiramate and one or more sympathomimetic agent coated beads can be present in the same dosage form. In active ingredients may include methylcellulose, sucrose, starch, microcrystalline cellulose, ethylcellulose, povidone, gelatin, talc, and titanium dioxide.

One or more dosage forms of topiramate and phentermine, for example, for use in one or more of the dosing regimens or methods described herein, such as the dosage forms described herein, can be packaged into a convenient packaging for delivery to or use by one or more physicians, subjects, nurses, health-care professionals, etc. Such packaging can include one or more sealed containers, each containing one or more dosage forms of topiramate, such as the dosage forms described herein.

### EXAMPLE 1

Liver disease was evaluated in patients participating in a study where a combination of phentermine and topiramate (PHEN/TPM) was administered to obese human patients. It was observed that a combination of topiramate with phentermine resulted in beneficial effects on markers that correlate with NAFLD/NASH.

A 56-week randomized controlled trial was conducted to evaluate safety and efficacy of a combination of phentermine and controlled-release topiramate for weight loss in obese adults. Results from the study (CONQUER) are reported in Gadde et al. Lancet 377:1341-1352 (2011). There were 979, 488 and 981 patients analyzed in the study in the three arms: placebo, 7.5 mg phentermine with 46 mg topiramate (mid dose), or 15 mg phentermine with 92 mg topiramate (top dose), respectively. The results demonstrated that the combination is safe and effective for weight loss and the data were subsequently evaluated for markers that correlate with liver disease.

It was noted during the study that the treatment resulted in significant improvements in a number of other markers including blood pressure, waist circumference, concentrations of lipids, glycaemia and inflammatory markers such as high-sensitivity C-reactive protein and adiponectin (see table 2 from Gadde et al. 2011). It was also noted that improvements in risk factors were most pronounced in patients with pre-existing comorbid diseases. For example, in hypertensive patients, greater reductions in systolic blood pressure were noted with PHEN/TPM treatment than in patients without hypertension. Also notable was the observation that the reduction in triglycerides and increase in HDL-C were more pronounced in patients with hypertriglyceridaemia than in the overall sample.

It was further noted that risk markers that are associated with liver disease all showed significant improvements. In particular, triglyceride and HDL-C levels, and ALT, fasting glucose, fasting insulin, HOMA-IR and adiponectin were all improved upon treatment with PHEN/TPM. All of these markers each are known to correlate with liver disease.

In the study sample ALT (alanine transaminase) levels decreased by 4 and 3.3 units (mU/ML) for the mid and top doses and only 0.8 for the placebo at 56 weeks. As discussed above, elevation of ALT levels is a known marker for liver disease and may indicate inflammation or damage to liver cells. ALT levels were also analyzed in a second study where in addition to the mid and top dose a low dose of 3.75 mg phentermine and 23 mg of topiramate were analyzed over a 1 year period (Allison et al. Obesity (2011) 20, 330-342). When looking at patients in the upper quartile of baseline value for ALT, the placebo group saw a 9.2 unit drop in ALT, the low dose resulted in a drop of 11.9 units, but the mid and top doses saw decreases of 16.7 and 16.3 units which were both statistically and clinically significant compared to placebo. (p<0.0001). This observed reduction in ALT, a marker known to correlate with liver disease, indicates that liver disease may benefit from treatment with PHEN/TPM.

In the study by Gadde et al., changes in fasting triglycerides from baseline in patients with hypertriglyceridemia also demonstrated a significant improvement in the treatment groups compared to the placebo (p<0.001). The placebo group showed a decrease of 8.8 units (mg/dL) while the mid and top doses showed decreases of 24.1 and 25.6 units, respectively. The reductions were even greater when looking at patients in the upper quartile of baseline value for triglycerides with decreases of 10.2, 28.2 and 27.8 units for placebo, mid and top, respectively.

Significant improvements in glycemic indicators were also observed. Fasting insulin was reduced 19.4% from baseline for mid and 21.7% for top but increased 3.9% for placebo. Insulin resistance (HOMA-IR) was reduced from baseline by 18.8% for mid and 20.2% for top but increased 8.9% for placebo. Insulin sensitivity during OGTT increased 50.9% and 54.2% from baseline for mid and top doses respectively, but only 14.1% for placebo.

Fasting serum glucose for patients in the upper quartile of baseline value showed decreases of 8.2, 12.5 and 16.3 units (mg/dL) in placebo, mid and top doses respectively. The change from placebo to top dose has a p<0.05 vs placebo.

Adiponectin levels increased from baseline to week 56 by 1.5 and 2.2 units for mid and top dose but only 0.4 units for placebo (p<0.0001 vs. placebo for both mid and top). In a separate study conducted by Allison et al. (Obesity 20:330-342 (2012)) similar results were observed for adiponectin, −0.4, 0.6 and 2.7 μg/mL changes for placebo, low (3.75/23) and top doses.

It has previously been shown that patients with NASH have lower serum adiponectin levels than patients with NAFLD so the increased adiponectin levels observed in the obese patients studied here indicates that PHEN/TOP may also be used as a treatment for liver disease or to prevent progression of liver disease in at risk patients.

Homeostatic model assessment of insulin resistance (HOMA-IR) is widely used for the estimation of insulin resistance and was developed as a convenient alternative to the euglycemic clamp method (Mathews et al. Diabetologia 28:412-419 (1985)). It is calculated by multiplying fasting plasma insulin (FPI) in 82 lU/mL by fasting plasma glucose (FPG) in mmol/L then dividing by the constant 22.5 (Wallace et al. Diabetes Care 27:387-392). Insulin resistance has been associated with metabolic and hemodynamic alterations and higher cardio metabolic risk and inflammation. Although recommended threshold values for diagnosing a patient as having insulin resistance (IR) vary from study to study and in different populations, in general a value above 2.6 has been used to diagnose patients as IR (Ascaso et al. Diabetes Care 26:3320-3325 (2003). A mean decrease of 0.93 and 1.07 was observed in patients treated with mid and top dose PHEN/TOP, so a significant improvement.

ALT levels with placebo, mid dose and top dose showed a decrease from baseline to week 56 of 0.8, 4 and 3.3 mU/mL respectively (OB-303). For patients in the upper quartile of baseline values the ALT change from baseline to week 56 was a decrease of 9.2, 11.9, 16.7 and 16.3 mU/mL for placebo, low, mid and top doses respectively (1-year cohort). Fasting triglycerides changed from baseline at week 56 in patients with hypertriglyceridemia was a decrease of 8.8, 24.1 and 25.6 for placebo, mid and top dose, respectively. For patients in the upper quartile of baseline value the change was a decrease of 10.2, 28.2 and 27.8 for the same. Percent change from baseline in fasting insulin was 3.9, −19.4 and 021.7 with placebo, mid, and top doses of PHEN/TPM, respectively. Similar changes in insulin resistance, and insulin sensitivity during OGTT were 8.9, −18.8 and −20.2, and 14.1 50.9 and 54.2, respectively.

### EXAMPLE 2

Analysis of PHEN/TPM in STAM model mice. Having observed the improvements in markers that correlate with liver disease in patients treated with PHEN/TPM, animal studies in the STAM model for NAFLD/NASH were preformed to look more specifically at liver morphology and function following treatment with PHEN/TPM. The STAM model was selected because it allows a specific examination of the effects of drug treatment, in this example PHEN/TPM, on non-alcoholic steatohepatitis. The STAM model is a non-genetic animal model of human NASH with features of diabetic background, increased NAFLD activity score (NAS), perivenular and pericellular fibrosis in Zone 3, and a high incidence of hepatocellular carcinoma (Fujii M. et al., Med. Mel. Morphol., 2013;46: 141). The model is induced by a combination of chemical and dietary interventions in the mice. In this example, NASH was induced in 30 male mice by a single subcutaneous injection of 200 μg streptozotocin (STZ, Sigma-Aldrich, USA) solution 2 days after birth and feeding with a high fat diet (HFD, 57 kcal % fat, cat#: HFD32, CLEA Japan, Japan) after 4 weeks of age. The mice were treated with either vehicle, PHEN/TPM or Telmisartan. Telmisartan has been shown to have anti-NASH, -fibrosis and -inflammatory effects in STAM mice and was used as a positive control.

Phentermine (PHEN) and Topiramate (TPM) were weighed and dissolved in distilled water and 0.5% methylcellulose, respectively. Telmisartan (MICARDIS®) was purchased from Boehringer Ingelheim GmbH (Germany) and suspended in milli-Q water. Vehicle, PHEN and TPM were orally administered in a volume of 5 mL/kg. Telmisartan was orally administered in a volume of 10 mL/kg. PHEN was administered at a dose of 15 mg/kg once daily. TPM was administered at a dose of 100 mg/kg once daily. Telmisartan was administered at a dose of 5 mg/kg once daily.

C57BL16 mice (15-day-pregnant female) were obtained from Japan SLC (Japan) and were housed in accordance with the Japanese Pharmacological Society Guidelines for Animal Use. The animals were maintained in a SPF facility under controlled conditions of temperature (23±2° C.), humidity (45±10%), lighting (12-hour artificial light and dark cycles; light from 8:00to 20:00) and air exchange. A high pressure was maintained in the experimental room to prevent contamination of the facility. The animals were housed in TPX cages (CLEA Japan) with a maximum of 4 mice per cage. Sterilized Paper-Clean (Japan SLC) was used for bedding and replaced once a week.

Sterilized solid HFD was provided ad libitum, being placed in a metal lid on the top of the cage. Pure water was provided ad libitum from a water bottle equipped with a rubber stopper and a sipper tube. Water bottles were replaced once a week, cleaned and sterilized in an autoclave and reused. Mice were identified by numbers engraved on earrings. Each cage was labeled with a specific identification code.

RESULTS. Measurement of blood biochemistry. Non-fasting blood glucose was measured in whole blood using LIFE CHECK (EIDrA, Japan). For plasma biochemistry, blood samples were collected in polypropylene tubes with anticoagulant (Novo-Heparin; Mochida. Pharmaceutical, Japan) and centrifuged at 1.000×g for 15 minutes at 4° C. The supernatant was collected and stored at −80° C. until use. The plasma ALT (alanine aminotransferase) and AST (aspartate aminotransferase) levels were measured by FUJI DRI CHEM 7000 (FujiFilm, Japan) Measurement of liver triglyceride content Liver total lipid-extracts were obtained by Folch's method (Folch J. et al., J Biol. Chern. 1957;226: 497). Liver samples were homogenized in chloroform-methanol (2:1, v/v) and incubated overnight at room temperature. After washing with chloroform-methanol-water (8:4:3, v/v/v), the extracts were evaporated to dryness, and dissolved in isopropanol. Liver triglyceride levels were measured by Triglyceride E-test (Wako Pure Chemical Industries, Japan).

Histopathology analysis. For HE staining, sections were cut from paraffin blocks of liver tissue prefixed in Bouin's solution and stained with Lillie-Mayers Hematoxylin (Muto Pure Chemicals, japan) and eosin solution (Wako Pure Chemical industries). NAS was calculated according to the criteria of Kleiner (Kleiner D E. et al., Hepatology, 2005;41: 1313). To visualize collagen deposition, Bouin's fixed liver sections were stained using picro-Sirius red solution (Waldeck, Gelman)). To visualize macro- and microvesicular fat, sections were cut from frozen liver tissues embedded in Tissue-Tek O.C.T. compound (Sakura Finetek Japan, Japan), and stained with Oil Red 0 (SigmaAldrich). For immunohistochemistry, sections were cut from frozen liver tissues embedded in Tissue-Tek O.C.T. compound and fixed in acetone. Endogenous peroxidase activity was blocked using 0.03% H202 for 5 minutes, followed by incubation with Block Ace (Dainippon Sumitomo Pharma, Japan) for 10 minutes. The sections were incubated with a 200-fold dilution of anti-F4/80 antibody (BMA Biomedicals, Switzerland) 1 hour at room temperature. After incubation with secondary antibody (HRP-Goat anti-rat antibody, Invitrogen, USA), enzyme-substrate reactions were performed using 3,3′-diaminobenzidine/H202 solution (Nichirei, Japan). For quantitative analyses of fibrosis, inflammation and fat deposition areas, bright field images of Sirius red-stained, F4/80-immunostained and oil red-stained sections were captured around the central vein using a digital camera (DFC280; Leica, Germany) at 200-fold magnification, and the positive areas in 5 fields/section were measured using ImageJ software (National Institute of Health, USA).

Gene expression levels of a number of markers that have been shown to correlate with liver disease, were evaluated. Total RNA was extracted from liver samples using RNAiso (Takara Bio, Japan) and SV total RNA isolation kit (Promega, USA) according to the manufacturer's instructions. One /lg of RNA was reverse-transcribed using a reaction mixture containing 4.4 mM MgCb (F. Hoffmann-La Roche, Switzerland), 40 U RNase inhibitor (Toyobo, Japan), 0.5 mM dNTP (Promega), 6.28/lM random hexamer (Promega), 5× first strand buffer (Promega), 10 mM dithiothreitol (Invitrogen, USA) and 200 U MMLV-RT (Invitrogen) in a final volume of 20 IlL. The reaction was carried out for 1 hour at 37° C., followed by 5 minutes at 99° C. Real-time PCR was performed using real-time PCR DICE and SYBR premix Tag (Takara Bio). To calculate the relative mRNA expression level, the expression of each gene was normalized to that of reference gene 36B4 (gene symbol: RplpO). To express data as fold-change relative to the Vehicle-control group, the data were standardized so that average values of the Vehicle group become 1.0. PCR-primer sets are provided in Table 1.

TABLE 1
Primer sequences for gene expression analysis.

| Gene | Set ID | sequence |
|---|---|---|
| 36B4 | MA057856 | forward 5′-TTCCAGGCTTTGGGCATCA-3′ (SEQ ID NO: 1) |
| | | reverse 5′-ATGTTCAGCATGTTCAGCAGTGTG-3′ (SEQ ID NO: 2) |
| TNF-α | MA092347 | forward 5′-GGAGTAGACAAGGTACAACCCATC-3′ (SEQ ID NO: 3) |
| | | reverse 5′-TATGGCCCAGACCCTCACA-3′ (SEQ ID NO: 4) |
| TIMP-1 | MA098519 | forward 5′-TGAGCCCTGCTCAGCAMGA-3′ (SEQ ID NO: 5) |
| | | reverse 5′-GAGGACCTGATCCGTCCACAA-3′ (SEQ ID NO: 6) |
| Alpha-SMA | MA057911 | forward 5′-AAGAGCATCCGACACTGCTGAC-3′ (SEQ ID NO: 7) |
| | | reverse 5′-AGCACAGCCTGAATAGCCACATAC-3′ (SEQ ID NO: 8) |
| MMP-9 | MA031311 | forward 5′-GCCCTGGAACTCACACGACA-3′ (SEQ ID NO: 9) |
| | | reverse 5′-TTGGAMCTCACACGCCAGAAG-3′ (SEQ ID NO: 10) |
| Collagen Type 1 | MA075477 | forward 5′-CCAACAAGCATGTCTGGTT AGGAG-3′ (SEQ ID NO: 11) |
| | | reverse 5′-GCAATGCTGTTCTTGCAGTGGTA-3′ (SEQ ID NO: 12) |
| MCP-1 | MA066003 | forward 5′-GCATCCACGTGTTGGCTCA-3′ (SEQ ID NO: 13) |
| | | reverse 5′-CTCCAGCCTACTCATTGGGATCA-3′ (SEQ ID NO: 14) |

Statistical analyses were performed using Bonferroni Multiple Comparison Test on GraphPad Prism 4 (GraphPad Software, USA). P values<0.05 were considered statistically significant. In particular cases, a trend or tendency was assumed when a one-tailed t-test returned P values<0.05. Results were expressed as mean≅SD.A

The three treatment groups were as follows: Group 1: Vehicle: 10 NASH mice were orally administered vehicle [distilled water and 0.5% methylcellulose in a volume of 5 mL./kg, respectively] once daily from 5 to 11 weeks of age. Group 2: PHEN/TPM. 10 NASH mice were orally administered distilled water supplemented with PHEN at a dose of 15 mg/kg, and 0.5% methylcellulose supplemented with TPM at a dose of 100 mg/kg once daily from 5 to 11 weeks of age. Group 3: Telmisartan. 10 NASH mice were orally administered pure water supplemented with Telmisartan at a dose of 5 mg/kg once daily from 5 to 11 weeks of age.

The viability, clinical signs and behavior were monitored daily. Mice were observed for significant clinical signs of toxicity, moribundity and mortality approximately 60 minutes after each administration. The animals were sacrificed by exsanguination through direct cardiac puncture under ether anesthesia (Wako Pure Chemical Industries). During the treatment period, sever& mice died before reaching week 11 as follows; four out of 10 mice found dead in the PHEN/TPM group. Two out of 10 mice found dead in the Vehicle and Telmisartan groups. The lower number of mice surviving in the PHEN/TPM group made it difficult to make conclusions with high statistical confidence, but the trends were clearly observable.

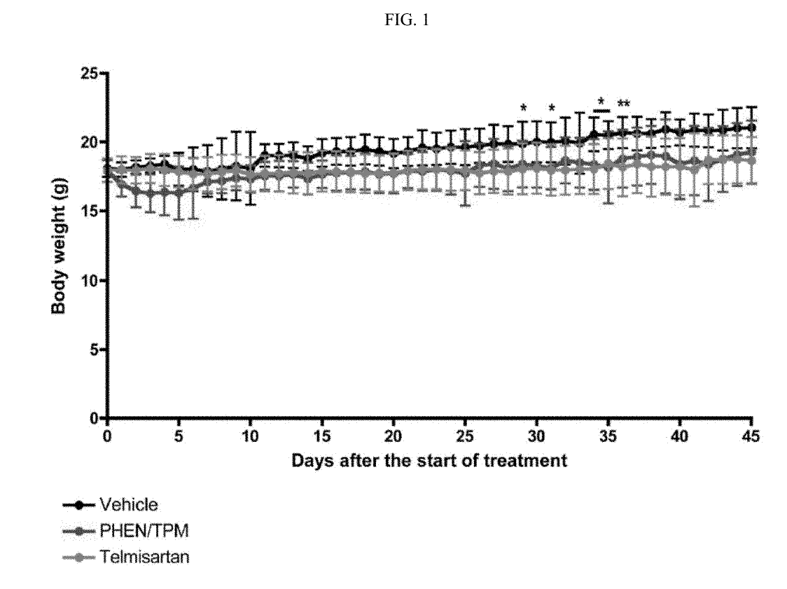

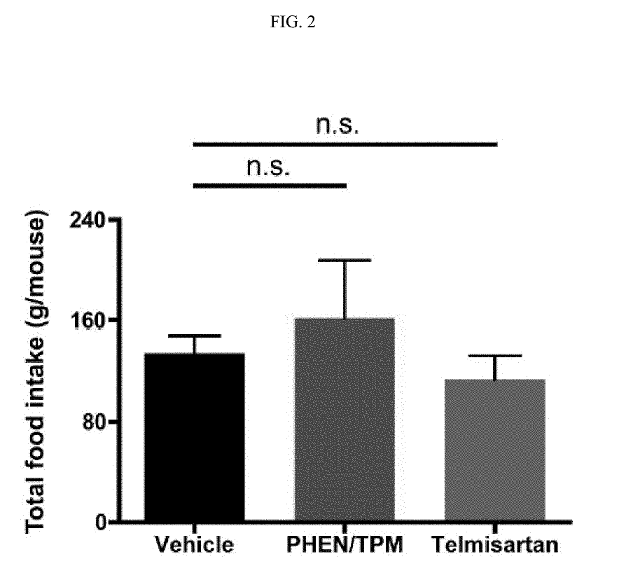

The body weight of each mouse was recorded daily and food consumption was recorded 3 times weekly during the treatment period. The results of body weight are shown in FIG. 1 and food consumption is shown in FIG. 2. Mean body weight gradually increased during the treatment period in the Vehicle group. Mean body weight in the PHEN/TPM and. Telmisartan groups did not show the same increase during the treatment period even though the PHEN/TPM mice consumed more food. The body weights in the Telmisartan and PHEN/TPM groups were lower than that in the vehicle group, particularly after about day 10. Body weight in the PHEN/TPM group tracked closely with those in the Telmisartan group.

Cumulative food intake over the life of the study was 135 g/animal in the vehicle group, 161 g/animal in the PHEN/TPM group, and 114 g/anitnal in the Telmisartan group (FIG. 2). Surprisingly the PHEN/TPM group consumed more food but still saw significant reduction in fat accumulation even though the diet being provided was a high fat diet. This supports a direct effect of PHEN/TPM in reducing fat accumulation and not simply that the animals consume less.

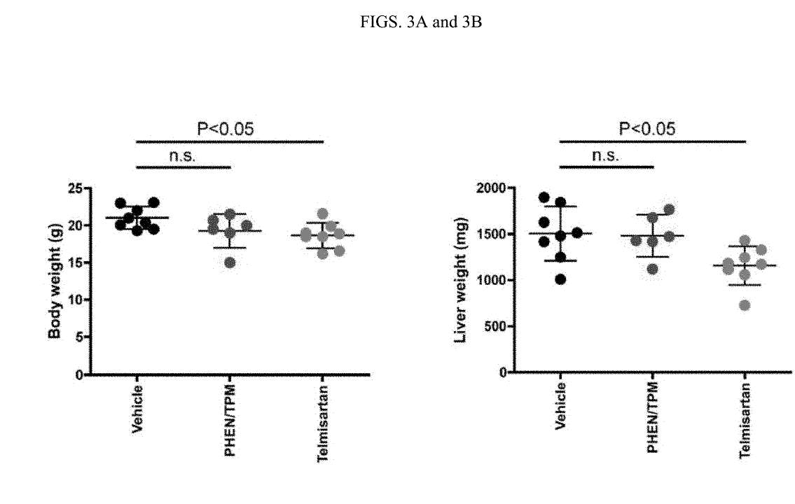

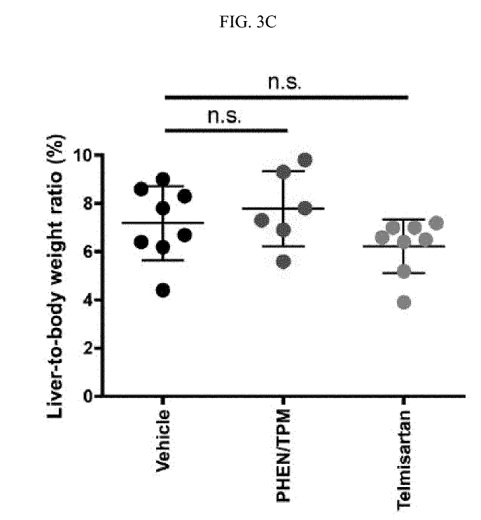

Body weight on the day of sacrifice is shown in FIG. 3A and in Table 2. Mean body weight on the day of sacrifice in the Telmisartan group was reduced by 11.4% compared to the Vehicle group (18.7 g vs. 21.1 g, respectively; p<0.05). Mean body weight was reduced by 8.5% in the PHEN/TPM group compared to the vehicle group (19.3 g vs. 21.1 g, respectively).

Liver weight is shown in FIG. 3B and liver-to-body weight ratio is graphed in FIG. 3C. The results are also provided in Table 2. Mean liver weights on the day of sacrifice were 1505 mg in the Vehicle group, 1158 mg in the telmisartan group and 1481 mg in the MEN/1′PM group. The liver weight in the telmisartan group was significantly lower than that in the Vehicle group. There was no significant difference in mean liver weight between the Vehicle group and the PHEN/TPM group and no significant difference in mean liver-to-body weight ratio between the Vehicle group and either of the treatment groups.

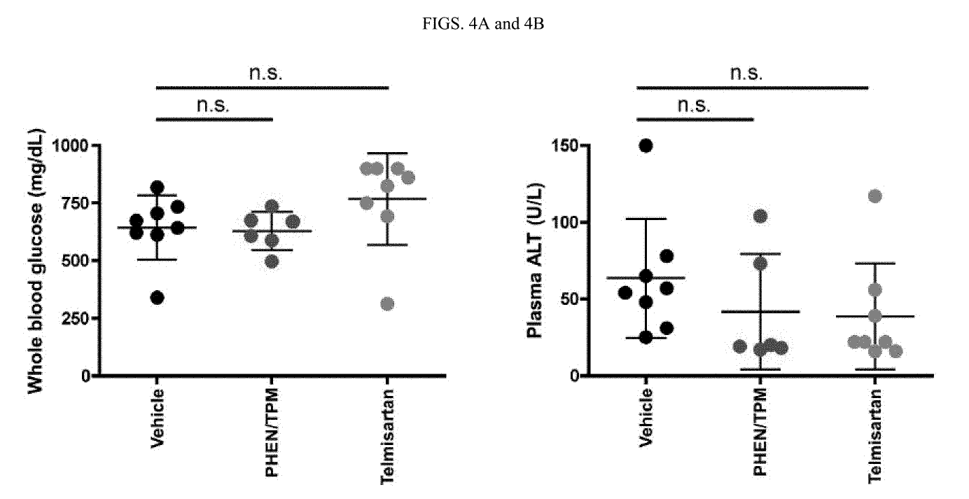

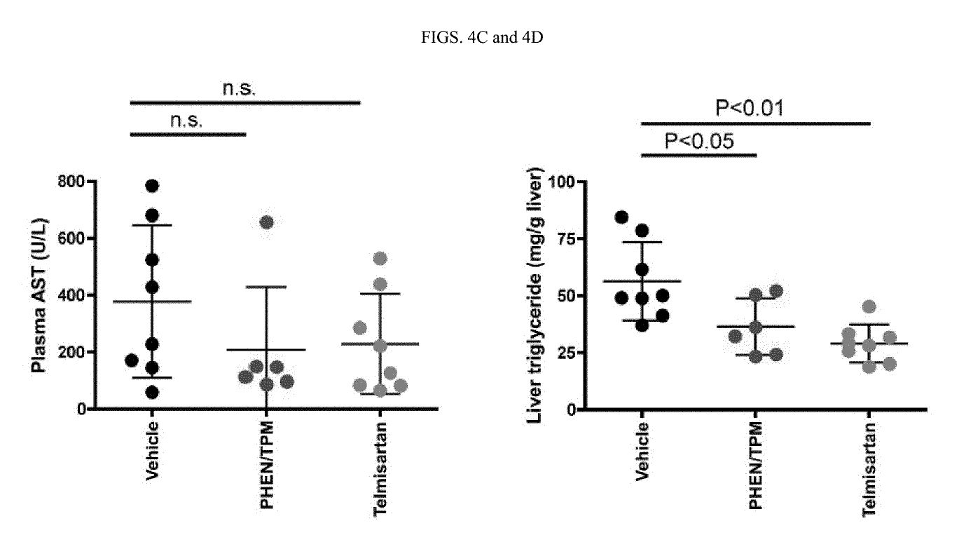

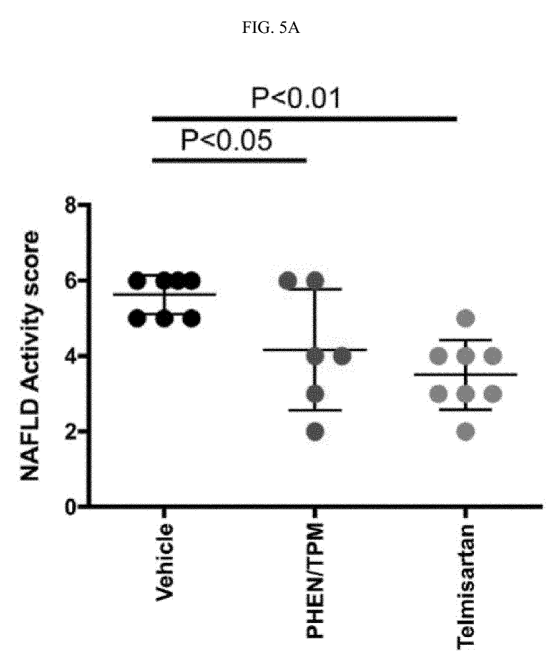

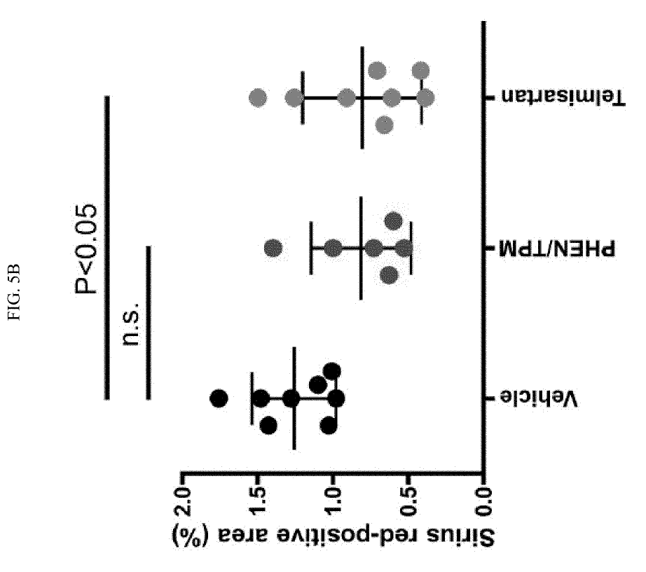

Mean blood glucose values on the day of sacrifice were 644 mg/dL in the Vehicle group, 768 mg/dL in the Telmisartan group, and 629 mg/dL in the PHEN/TPM group. The results are shown graphically in FIG. 4A. The whole blood glucose levels in the PHEN/TPM between the Vehicle group and any of the treatment groups. The levels in the Telmasartin group are noticeably higher, but the PHEN/TPM mice show levels similar to the vehicle. Given that the PHEN/TPM group consumed more food and consequently more fat than the other two groups it is notable that the glucose levels did not go up in the PHEN/TPM group. This suggests that insulin sensitivity is increasing as a result of PHEN/TPM treatment, suggesting a protective effect for liver function resulting from PHEN/TPM treatment. Insulin resistance and insulin levels are triggers for NASH and result in increased liver fat deposition along with other indicators of NASH.

Plasma ALT levels are graphed in FIG. 4B and shown in Table 3. Mean ALT values on the day of sacrifice were 64 U/L in the Vehicle group, 39 U/L in the Telmisartan group, and 42 U/L in the PHEN/IPM group. FIG. 4B shows that the plasma ALT levels of the vehicle group are higher than the levels of both treatment groups. Providing further indication that PHEN/TPM has a beneficial effect on prevention of liver disease.

TABLE 2
Body weight and liver weight

| Parameter | Vehicle | PHEN/TPM | Telmisartan |
|---|---|---|---|
| (mean ± SD) | (n = 8) | (n = 6) | (n = 8) |
| Body weight (g) | 21.1 ± 1.5 | 19.3 ± 2.3 | 18.7 ± 1.7 |
| Liver weight (mg) | 1505 ± 294 | 1481 ± 226 | 1158 ± 210 |
| Liver-to-body weight ratio (%) | 7.2 ± 1.5 | 7.8 ± 1.6 | 6.2 ± 1.1 |

Plasma AST levels are shown in FIG. 4C and in Table 3. Mean AST values on the day of sacrifice were 378 U/L in the Vehicle group, 229 U/L in the Telmisartan group, and 208 U/L in the PHEN/TPM group. The plasma AST levels of the vehicle group are higher than either treatment groups. Both the ALT and AST markers show a clear indication that PHEN/TPM treatment provides protection against liver cell damage. Each of the markers analyzed in the study that are associated with liver damage trended in the same direction for PHEN/TPM group as for the positive control. These data support the conclusion that PHEN/TPM treatment is effective for treating liver disease in mice with established NASH.

TABLE 3
Biochemistry

| Parameter | Vehicle | PHEN/TPM | Telmisartan |
|---|---|---|---|
| (mean ± SD) | (n = 8) | (n = 6) | (n = 8) |
| Whole blood glucose (mg/dL) | 644 ± 140 | 629 ± 83 | 768 ± 199 |
| Plasma ALT (U/L) | 64 ± 39 | 42 ± 37 | 39 ± 34 |
| Plasma AST (U/L) | 378 ± 268 | 208 ± 221 | 229 ± 176 |
| Liver triglyceride (mg/g liver) | 56.4 ± 17.2 | 36.4 ± 12.5 | 29.0 ± 8.3 |

Liver triglyceride levels are shown in (FIG. 4D and Table 3). Mean liver triglyceride content values on the day of sacrifice were 56.4 mg/g liver in the Vehicle group, 29.0 mg/g liver in the Telmisartan group, and 36.4 mg/g liver in the PHEN/TPM group. Liver triglyceride levels decreased significantly in both the PHEN/TPM and the Telmisartan groups compared with the Vehicle group. The numbers are striking given that the PHEN/TPM mice were eating more food and more fat than both of the other groups.

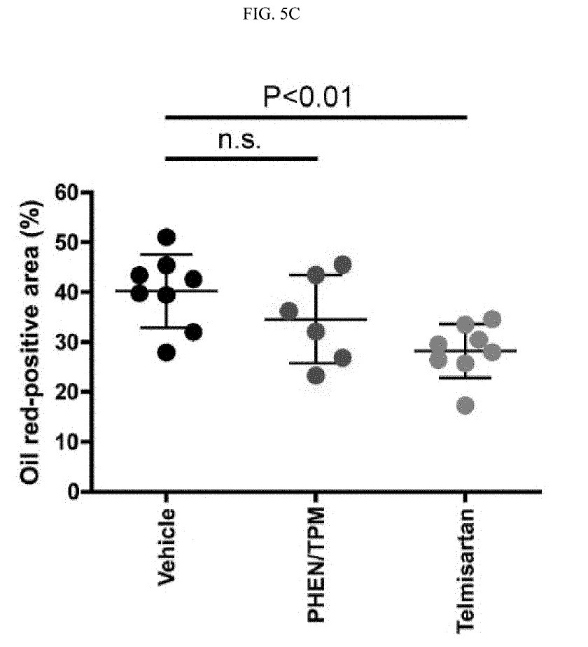

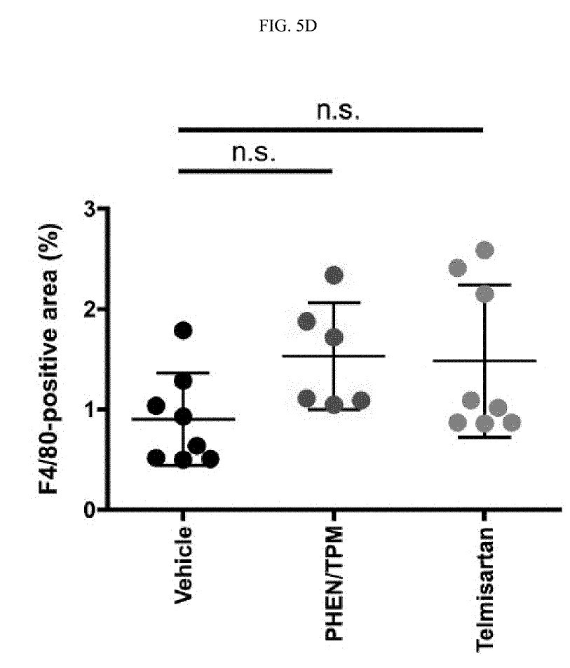

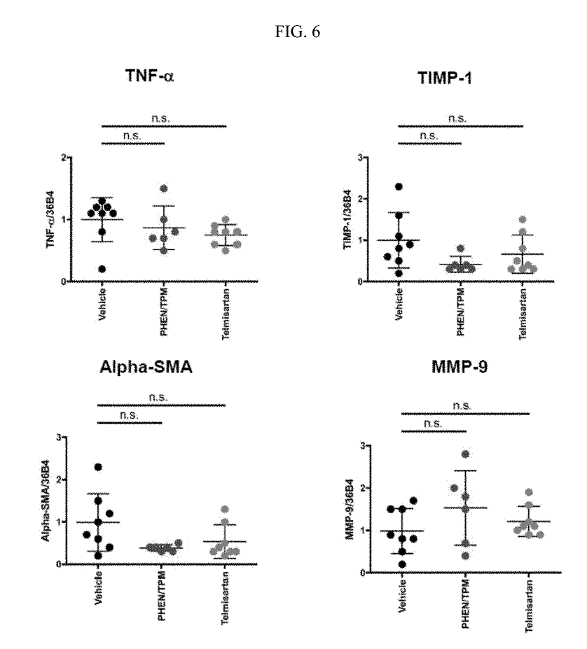

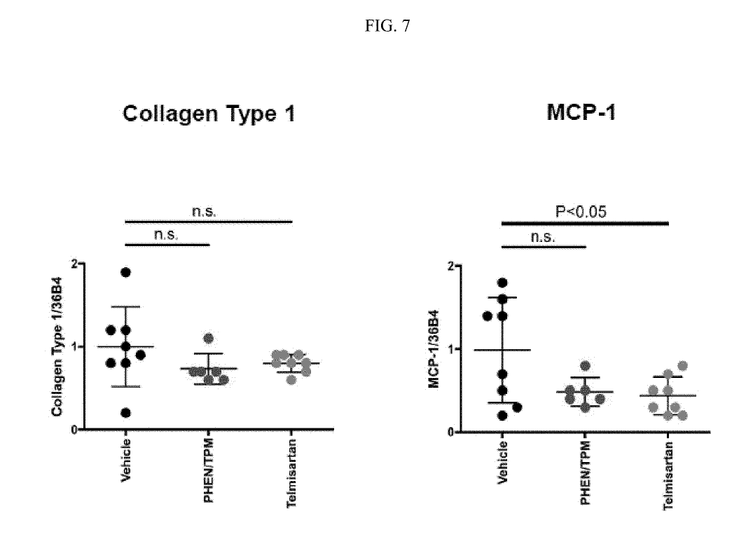

NAFLD Activity scores are shown in FIG. 5A and Table 4. Haematoxylin Eosin staining, or “HE-staining” was performed and HE-stained sections were examined. Liver sections from the Vehicle group exhibited severe micro- and macrovesicular fat deposition, hepatocellular ballooning and inflammatory cell infiltration. The PHEN/TPM and the Telmisartan groups showed a significant reduction in NAS compared with the Vehicle group. The NAFLD Activity score used in this study is the same scoring system that is used as a primary endpoint in NASH studies in humans. A 2 point reduction in NAFLD activity score is generally viewed as clinically significant.

TABLE 4
NAFLD Activity scores.

| | Score | | | | | | | | | | | | |
|---|---|---|---|---|---|---|---|---|---|---|---|---|---|
| | | Steatosis | | | | Lobular inflammation | | | | Hepatocyte ballooning | | | NAS |
| Group | n | 0 | 1 | 2 | 3 | 0 | 1 | 2 | 3 | 0 | 1 | 2 | (mean ± SD) |
| Vehicle | 8 | — | 8 | — | — | — | — | 3 | 5 | — | — | 8 | 5.6 ± 0.5 |
| PHEN/TPM | 6 | — | 6 | — | — | 1 | 1 | 1 | 3 | 1 | 3 | 2 | 4.2 ± 1.6 |
| Telmisartan | 8 | 2 | 6 | — | — | 2 | 2 | 4 | — | — | 4 | 4 | 3.5 ± 0.9 |

TABLE 5
Definition of NAS Components

| Item | Score | Extent |
|---|---|---|
| Steatosis | 0 | <5% |
| | 1 | 5-33% |
| | 2 | >33-66% |
| | 3 | >66% |
| Hepatocyte | 0 | None |
| Ballooning | 1 | Few balloon cells |
| | 2 | Many cells/prominent ballooning |
| Lobular | 0 | No foci |
| Inflammation | 1 | <2 foci/200x |
| | 2 | 2-4 foci/200x |
| | 3 | >4 foci/200x |

Sirius red-stained sections of livers were also analyzed. Morphometrically quantified liver collagen (Sirius Red) serum hyaluronic acid are both known to be elevated in histological NASH versus NAFLD. The results are shown in Table 6. Liver sections from the Vehicle group exhibited collagen deposition in the peri-central region of liver lobule, with a total fibrosis area of 1.26%. Fibrosis areas in the Telmisartan and PHEN/TPM groups were 0.81% and 0.82%, respectively. Both the Telmisartan group and the PHEN/TPM group showed reductions in fibrosis area compared to the Vehicle group.

Oil red-stained sections of livers were analyzed and the results are shown in Table 6. In liver sections, the fat deposition area, as determined by oil red staining was 40.24% in the Vehicle group, 28.24% in the Telmisartan group, and 34.60% in the PHEN/TPM group. The Telmisartan group showed a significant reduction in fat deposition area compared with the Vehicle group. The difference in the fat deposition area between the Vehicle group and the PHEN/TPM group was not clear, but the observed trend was consistent with a beneficial effect. F4/80 immunohistochemistry staining of liver sections was also analyzed and the results are reported in Table 6.

The inflammation area appeared to be increasing in both the PHEN/TPM and the telmasartin groups compared to the Vehicle group, so again the PHEN/TPM treatment was behaving like the positive control, although it is not clear how this effect on inflammation might be beneficial.

TABLE 6
Histological analysis

| Parameter | Vehicle | PHEN/TPM | Telmisartan |
|---|---|---|---|
| (mean ± SD) | (n = 8) | (n = 6) | (n = 8) |
| Fibrosis area (Sirius-red) | 1.26 ± 0.28 | 0.82 ± 0.33 | 0.81 ± 0.39 |
| (%) | | | |
| Fat deposition area (Oil- | 40.24 ± 7.36 | 34.60 ± 8.90 | 28.24 ± 5.40 |
| red) (%) | | | |
| Inflammation area (F4/80) | 0.90 ± 0.46 | 1.53 ± 0.53 | 1.48 ± 0.76 |
| (%) | | | |

GENE EXPRESSION ANALYSIS. Changes in the expression levels of a number of genes have been shown to correlate with liver disease. To determine the effect of PHEN/TPM on the expression of these markers mRNA analysis was performed for a collection of genes previously identified as being associated with liver disease. Tumor necrosis factor alpha (TNF-α) is known to play an important role in insulin resistance by inhibiting the tyrosine kinase activity of the insulin receptor. It has been reported that patients with NASH have higher TNF-α and its soluble receptor (sTNFR1) than those with simple steatosis (see Abiru et al. Liver Int 2006: 26:39-45). It has also been reported that patients with NASH have higher levels of TNF-α mRNA (see Abiru et al. Liver Int 2006: 26:39-45 and Alaaeddin et al. Eur Cytokine Netw 2012, 23:107-111). The mean fold changes of TNF-α mRNA expression levels were 0.87 in the PHEN/TPM group and 0.75 in the Telmisartan group relative to the Vehicle group. The TNF-α mRNA expression levels were higher in the treatment groups than in the vehicle.

The mRNA levels of 6 different genes were analyzed, TNF-a (tumor necrosis factor), TIMP-1 (tissue inhibitor of metalloproteinase 1), Alpha-SMA [Actin, alpha 2, smooth muscle, aorta], MMP-9 (Matrix metallopeptidase 9), collagen type 1 [Collagen, type I, alpha 2 (CoI1a2)] and MCP-1 [Chemokine (C-C motif) ligand 2 (CcI2)]. All were normalized using ribosomal protein 36B4 as a control. For both PHEN/TPM and telmisartan expression of 5 of the 6 decreased and expression of MMP-9 increased. The normalized values are shown in Table 7.

For TIMP-I the mean fold changes of TIMP-1 mRNA expression levels were 0.42 in the PHEN/TPM group and 0.66 in the Telmisartan group relative to the Vehicle group. The mean fold changes of Alpha-SMA mRNA expression levels were 0.38 in the PHEN/TPM group and 0.54 in the Telmisartan group relative to the Vehicle group. The mean fold changes of MMP-9 mRNA expression levels were 1.53 in the PHEN/TPM group and 1.21 in the Telmisartan group relative to the Vehicle group. The mean fold changes of Collagen Type 1 mRNA expression levels were 0.73 in the PHEN/TPM group and 0.80 in the Telmisartan group relative to the Vehicle group. The mean fold changes of MCP-I mRNA expression levels were 0.48 in the PHEN/TPM group and 0.44 in the Telmisartan group relative to the Vehicle group.

The values in Table 7 were calculated by dividing Target by 36B4 for each animal. An average was calculated in the vehicle group. The initial calculations of Target/36B4 was divided by the vehicle average so the vehicle average becomes 1.0. This second. Target/36B4 represents a fold-change of Target gene relative to the vehicle average.

The results of the gene expression analysis are consistent with PHEN/TPM treatment having the same effect as teltnisartan. Each marker that was expected to go down was observed to go down and the 1 marker that was expected to go up was observed to increase, all as would be expected for a reduction in fibrosis.

TABLE 7
Gene expression analyses

| Parameter | Vehicle | PHEN/TPM | Telmisartan |
|---|---|---|---|
| (mean ± SD) | (n = 8) | (n = 6) | (n = 8) |
| TNF-α/36B4 | 1.00 ± 0.35 | 0.87 ± 0.35 | 0.75 ± 0.17 |
| TIMP-1/36B4 | 1.00 ± 0.67 | 0.42 ± 0.19 | 0.66 ± 0.46 |
| Alpha-SMA/36B4 | 1.00 ± 0.68 | 0.38 ± 0.08 | 0.54 ± 0.40 |
| MMP-9/36B4 | 1.00 ± 0.53 | 1.53 ± 0.88 | 1.21 ± 0.36 |
| Collagen Type 1/36B4 | 1.00 ± 0.48 | 0.73 ± 0.19 | 0.80 ± 0.11 |
| MCP-1/36B4 | 1.00 ± 0.63 | 0.48 ± 0.17 | 0.44 ± 0.23 |

Telmisartan has been shown to have anti-inflammatory and anti-fibrotic effects in SIAM mice presumably through its angiotensin receptor antagonist activity, and anti-steatosis effect through a PPAR γ/α agonist activities. In historical data, Telmisartan has consistently demonstrated reductions in liver weight, NAS and the fibrosis area, and has shown trends toward decreased body weight, the body-to-liver weight ratio, liver TG, and hepatic expression levels of several inflammatory and fibrotic genes. Telmisartan-receiving SIAM mice did not show significant changed in food intake, thus the body weight loss seen in the Telmisartan group is probably due to another cause such as increase in energy expenditure (Sugimoto K., et al., Hypertension, 2006;47: 1003). In the present study, treatment with Telmisartan significantly decreased NAS and fibrosis area compared with the Vehicle group, validating the use of the drug as a positive control.

Treatment with PHEN/TPM decreased liver triglyceride and NAS with statistical significance, and showed a decrease in the fibrosis area and the expression levels of a-SMA and MCP-1 in the present study. The improvement of NAS is important because it is one of the major endpoints for assessing drug efficacy in NASH patients (Sanyal A J. et al., Hepatology, 2011;54:344), and thus is a useful non-clinical endpoint in clinical translation. PHEN/TPM also showed efficacy in liver fibrosis of SIAM mice. The decreasing trend in collagen gene expression supported potential anti fibrotic effects of PHEN/TPM.

From a mechanistic point of view, a noteworthy observation in the PHEN/TPM group was the decrease in liver lipid (triglyceride). Hepatic fat accumulation is the classical “first hit” of NAFLD and thought to be prerequisite for hepatocyte injury, subsequent induction of pro-inflammatory/fibrotic gene responses, and ultimately the progression to advanced NASH or hepatocellular carcinoma (HCC). The observed changes in the genes expression levels could be attributed to amelioration of steatosis by PHEN/TPM, which may have then played a role in delaying the disease progression of NAFLD and led to the histological improvements achieved in this study.

We claim:

1. A method of treating or preventing the progression of liver disease in a patient in need thereof, comprising administering to the patient a therapeutically effective amount of topiramate in combination with a therapeutically effective amount of phentermine.

2. The method of claim 1, wherein the liver disease is selected from the group consisting of chronic liver disease, metabolic liver disease, steatosis, liver fibrosis, primary sclerosing cholangitis (PSC), cirrhosis, liver fibrosis, non-alcoholic fatty liver disease (NAFLD), non-alcoholic steatohepatitis (NASH), hepatic ischemia reperfusion injury, primary biliary cirrhosis (PBC), and hepatitis.

3. The method of claim 1, wherein the topiramate is a compound of formula (I): or a pharmaceutically acceptable salt, isomer, stereoisomer, or tautomer thereof and the phentermine is a compound of formula (II): or a pharmaceutically acceptable salt, isomer, stereoisomer, or tautomer thereof.

4. The method of claim 1, wherein the topiramate and the phentermine are administered orally in a single dosage form that is administered once daily.

5. The method of claim 1, wherein the topiramate is administered at a daily dose of 23, 46, 69, or 92 mg and the phentermine is administered at a daily dose of 3.75, 7.5 11.25 or 15 mg.

6. The method of claim 5, wherein the 3.75, 7.5, 11.25 and 15 mg of phentermine is provided as 4.67, 9.33, 14 and 18.67 mg phentermine hydrochloride.

7. The method of claim 5, wherein the patient is administered a first daily dose of 23 mg topiramate and 3.75 mg phentermine for 1 to 3 weeks followed by a second daily dose of 46 mg topiramate and 7.5 mg phentermine for at least 10 weeks.

8. The method of claim 7, wherein the second daily dose is administered for at least 56 weeks.

9. The method of claim 1 wherein the patient has a body mass index of at least 27 kg/m².

10. The method of claim 1 wherein the patient has a body mass index of at least 30 kg/m².

11. A method of treating or slowing the progression of liver disease in a patient in need thereof, comprising administering to the patient a therapeutically effective amount of topiramate in combination with a therapeutically effective amount of phentermine, wherein the liver disease is non-alcoholic steatohepatitis (NASH) and wherein the therapeutically effective amount of topiramate is selected from 46 mg per day and 92 mg per day and the therapeutically effective amount of phentermine is selected from 7.5 mg per day and 15 mg per day wherein the 7.5 mg phentermine is provided as 9.33 mg phentermine hydrochloride and the 15 mg phentermine is provided as 18.66 mg phentermine hydrochloride.

12. A method of delaying the progression from NAFLD to NASH in a patient, comprising: identifying a patient having NAFLD and administering to the patient an oral dosage form comprising immediate release phentermine and controlled release topiramate.

13. The method of claim 12 wherein the oral dosage form comprises: a) 3.75 mg immediate release phentermine in combination with 23 mg controlled release topiramate, orb) 7.5 mg immediate release phentermine in combination with 46 mg controlled release topiramate, or c) 11.25 mg immediate release phentermine in combination with 69 mg controlled release topiramate, or d) 15 mg immediate release phentermine in combination with 92 mg controlled release topiramate.

14. The method of claim 13 wherein the oral dosage form is administered to the patient for at least 3 months.

15. The method of claim 12 wherein a first oral dosage form is administered to the patient for 1 to 2 weeks and a second oral dosage form is administered to the patient for 3 months and wherein the first oral dosage form comprises 3.75 mg immediate release phentermine in combination with 23 mg controlled release topiramate and the second oral dosage form comprises 7.5 mg immediate release phentermine in combination with 46 mg controlled release topiramate.

16. The method of claim 12 wherein the step of identifying a patient having NAFLD comprises determining that the patient has at least one of the following: (i) hepatic accumulation of triglycerides in the hepatocytes in the absence of clinically significant alcohol intake; (ii) simple hepatic steatosis; (iii) fat making up more than 10% of the weight of the liver, and (iii) fibrosis.

17. The method of claim 12 further comprising continuing to administer the oral dosage form to the patient following the onset of NASH and thereby reducing the severity of NASH symptoms in the patient.

18. The method of claim 14 wherein the administering prevents the onset of NASH in the patient for the at least 3 months.

19. The method of claim 18 wherein the progression of NAFLD to NASH is prevented if the patient has at least one of the following after the at least 3 months: (i) percentage of liver weight that is contributed by fat has not increased since starting the administration; (ii) the fibrotic area of the liver has not increased since starting the administration (iii) the inflammation area of the liver has not increased since starting administration and (iv) NAFLD activity score is less than 5.

20. The method of claim 12 further comprising achieving in the patient a reduction in the patient's NAFLD activity score to less than 5.

21. The method of claim 12 further comprising achieving in the patient an improvement in at least one of the following: a decreased NAFLD activity score, decreased plasmas AST, decreased liver tryiglyceride levels, decreased plasma ALT, and decreased whole blood glucose.

22. The method of claim 12 further comprising achieving in the patient an improvement in at least one of the following over the measurements for the patient prior to administering the oral dosage form to the patient: a decreased NAFLD activity score, decreased plasmas AST, decreased liver tryiglyceride levels, decreased plasma ALT, and decreased whole blood glucose.

23. The method of claim 12 wherein the patient is identified as potentially having NAFLD and being at risk to progress to NASH if the patient is obese, has type 2 diabetes or insulin resistance.

24. A method of delaying the progression from NASH to liver cirrhosis in a patient, comprising: identifying a patient having NASH and administering to the patient an oral dosage form comprising: a) 3.75 mg immediate release phentermine in combination with 23 mg controlled release topiramate, or b) 7.5 mg immediate release phentermine in combination with 46 mg controlled release topiramate, or c) 11.25 mg immediate release phentermine in combination with 69 mg controlled release topiramate, or d) 15 mg immediate release phentermine in combination with 92 mg controlled release topiramate.


SEQUENCE LISTING (14 sequences)

SEQ ID NO: 1 - DNA, 19 bp, Artificial Sequence
  Synthetic Polynucleotide
ttccaggctttgggcatca

SEQ ID NO: 2 - DNA, 24 bp, Artificial Sequence
  Synthetic Polynucleotide
atgttcagcatgttcagcagtgtg

SEQ ID NO: 3 - DNA, 24 bp, Artificial Sequence
  Synthetic Polynucleotide
ggagtagacaaggtacaacccatc

SEQ ID NO: 4 - DNA, 19 bp, Artificial Sequence
  Synthetic Polynucleotide
tatggcccagaccctcaca

SEQ ID NO: 5 - DNA, 19 bp, Artificial Sequence
  Synthetic Polynucleotide
tgagccctgctcagcamga

SEQ ID NO: 6 - DNA, 21 bp, Artificial Sequence
  Synthetic Polynucleotide
gaggacctgatccgtccacaa

SEQ ID NO: 7 - DNA, 22 bp, Artificial Sequence
  Synthetic Polynucleotide
aagagcatccgacactgctgac

SEQ ID NO: 8 - DNA, 24 bp, Artificial Sequence
  Synthetic Polynucleotide
agcacagcctgaatagccacatac

SEQ ID NO: 9 - DNA, 20 bp, Artificial Sequence
  Synthetic Polynucleotide
gccctggaactcacacgaca

SEQ ID NO: 10 - DNA, 21 bp, Artificial Sequence
  Synthetic Polynucleotide
ttggamctcacacgccagaag

SEQ ID NO: 11 - DNA, 24 bp, Artificial Sequence
  Synthetic Polynucleotide
ccaacaagcatgtctggttaggag

SEQ ID NO: 12 - DNA, 23 bp, Artificial Sequence
  Synthetic Polynucleotide
gcaatgctgttcttgcagtggta

SEQ ID NO: 13 - DNA, 19 bp, Artificial Sequence
  Synthetic Polynucleotide
gcatccacgtgttggctca

SEQ ID NO: 14 - DNA, 23 bp, Artificial Sequence
  Synthetic Polynucleotide
ctccagcctactcattgggatca